(19) United States
(12) Patent Application Publication
(10) Pub. No.: US 2012/0283219 A1
(43) Pub. Date: Nov. 8, 2012

(54) DIAMINOPTERIDINE DERIVATIVES

(76) Inventors: Philip D.G. Coish, North Haven, CT (US); Phil Wickens, Richmond Hill (CA); Brian R. Dixon, Woodbridge, CT (US); David Osterman, Glastonbury, CT (US); Uday R. Khire, Orange, CT (US); Manuel Navia, Boston, MA (US); Judd Berman, Toronto (CA); Harpreet Kaur, Brampton (CA); Jeffrey Wilson, Toronto (CA); Dennis Underwood, Jamaica Plain, MA (US)

(21) Appl. No.: 13/257,325

(22) PCT Filed: Mar. 25, 2010
(86) PCT No.: PCT/US2010/000904

§ 371 Date: Nov. 21, 2011

Related U.S. Application Data
(60) Provisional application No. 61/211,137, filed on Mar. 25, 2009.

(51) Int. Cl. A61K 31/519; C07F 9/6561; C07D 403/12; C07F 9/6512; A61P 1/12; A61P 31/00; A61P 31/04; A61P 31/10; A61P 13/02; C07D 475/08; A61K 31/675

(52) U.S. Cl. 514/81; 544/260; 544/244; 544/296; 544/243; 514/249

(57) ABSTRACT

The present invention relates novel diaminopteridine derivatives, their compositions and method of treatment comprising the same for use as anti-infectives.

This application claims priority from U.S. Provisional Application No. 61/211,137, filed Mar. 25, 2009, the contents of which are incorporated by reference in their entirety.

### TECHNICAL FIELD

The present invention relates to diaminopteridine derivatives and their compositions for use as anti-infectives.

### BACKGROUND OF THE INVENTION

The fast growing rate of antibiotic resistance over the past decades has raised serious concerns that the antibiotic treatment options currently available will soon be ineffective. With the widespread usage of antibiotics in combination with the rapid growing rate of bacterial resistance in stark contrast with the decade-old chemical scaffolds available for their treatment, it is imperative that new drugs are developed in the battle against bacterial pathogens.

Anti-microbial agents may act via a number of known mechanisms, e.g., by inhibiting synthesis of bacterial cell walls, by acting directly on cell membrane of the microorganism to increase permeability and leakage of intracellular compounds, by disrupting function of various ribosomal subunits to reversibly inhibit protein synthesis, by binding to various ribosomal subunit and alter protein synthesis, by affecting bacterial nucleic acid metabolism or by blocking essential enzymes of folate metabolism. In the recent years, studies have shown that RNA structures in many bacteria and fungi termed riboswitches may regulate the expression of various genes crucial for survival or virulence. Typically located within the 5′-untranslated region (5′-UTR) of certain mRNAs, members of each known class of riboswitch can fold into a distinct, three-dimensionally structured receptor that recognizes a specific organic metabolite. When the cognate metabolite is present at sufficiently high concentrations during transcription of the mRNA, the riboswitch receptor binds to the metabolite and induces a structural change in the nascent mRNA that prevents expression of the open reading frame (ORF), thereby altering gene expression. In the absence of the cognate metabolite, the riboswitch folds into a structure that does not interfere with the expression of the ORF.

Sixteen different classes of riboswitches have been reported. Members of each class of riboswitch bind to the same metabolite and share a highly conserved sequence and secondary structure. Riboswitch motifs have been identified that bind to thiamine pyrophosphate (TPP), flavin mononucleotide (FMN), glycine, guanine, 3′-5′-cyclic eiguanylic acid (c-di-GMP), molybdenum cofactor, glucosamine-6-phosphate (GlcN6P), lysine, adenine, and adocobalamin (AdoCbl) riboswitches. Additionally, four dinstinct riboswitch motifs have been identified that recognize S-adenosylmethionine (SAM) I, II and III, IV and two distinct motifs that recognize pre-queosine-1 (PreQ1). Several antimetabolite ligands have also been identified that bind to known riboswitch classes, including pyrithiamine pyrophosphate (PTPP) which binds TPP riboswitches, L-aminoethylcysteine (AEC) and DL-4-oxalysine which bind to lysine riboswitches and roseoflavin and FMN which bind to FMN riboswitches. The riboswitch-receptors bind to their respective ligands in an interface that approaches the level of complexity and selectivity of proteins. This highly specific interaction allows riboswitches to discriminate against most intimately related analogs of ligands. For instance, the receptor of a guanine-binding riboswitch from Bacillus subtilis forms a three-dimensional structure such that the ligand is almost completely enveloped. The guanine is positioned between two aromatic bases and each polar functional group of the guanine hydrogen bonds with four additional riboswitch nucleotides surrounding it. This level of specificity allows the riboswitch to discriminate against most closely related purine analogs. Similarly, studies of the SAM-binding riboswitches reveal that nearly every functional group of SAM is critical in binding the ligands, allowing it to discriminate highly similar compounds such as S-adenosylhomocysteine (SAH) and S-adenosylmethionine (SAM), which only differ by a single methyl group. Likewise, TPP riboswitches comprise one subdomain that recognizes the polar functional group of the 4-amino-5-hydroxymethyl-2-methylpyrimidine (HMP) moiety and another subdomain that coordinates two metal ions and several water molecules to bind the negatively charged pyrophosphate moiety of the ligand. Similar to TPP, guanine and SAM riboswitches, FMN riboswitches form receptor structures that are highly specific for the natural metabolite FMN. It is by this highly specific interaction that allows for the design of small molecules for the regulation of specific genes.

The riboswitch that is of particular interest is the TPP riboswitch. TPP is an essential cofactor in bacteria, archaea, and eukaryotes. Organisms such as bacteria, plants and fungi, use TPP-sensing riboswitches to control genes responsible for importing or synthesizing thiamine and its phosphorylated derivatives. Studies have shown that binding of ligand to the 5′-untranslated region of E. Coli thiM gene, which is involved in the biosynthesis of thiamine, turns the riboswitch structure “off” and reduces translation of the mRNA by sequestering the ribosome binding site. Similar to bacterial riboswitch, eukaryotic riboswitches bind TPP with a similar affinity and undergo the same conformational changes. As such, compounds that target TPP riboswitch may be used to regulate or inhibit biosynthesis of thiamine and its phosphorylated derivatives necessary for many cellular processes.

It is therefore the objective of this invention to provide compounds useful for treating an infection, particularly compounds that target TPP riboswitch.

### SUMMARY OF THE INVENTION

In the first aspect, the invention provides a Compound of Formula Q-I:

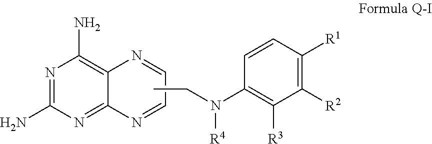

wherein
(i) R¹, R² and R³ are as follows:
   (a) R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
   (b) R² and R³ are independently COOR⁵; or
   (c) R² and R³ are independently —OH or —OCH₂COOR⁵ and R¹ is —COOR⁵;
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
   in free, salt or prodrug form.

In another embodiment, the invention provides a Compound of Formula Q-II:

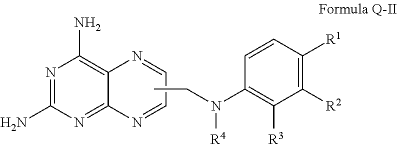

wherein
(i) R¹, R² and R³ are as follows:
   (a) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵ or
   (b) R² and R³ are independently —OH or —OCH₂COOR⁵ and R¹ is —COOR⁵;
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl),
(vi) Rₐ and R_{b} are independently H or C₁₋₄alkyl,
   in free, salt or prodrug form.

In another embodiment, the invention provides a Compound of Formula Q-III:

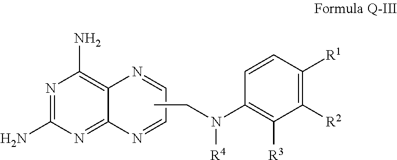

wherein
(i) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
   in free, salt or prodrug form.

In another embodiment, the invention provides a Compound of Formula Q-IV:

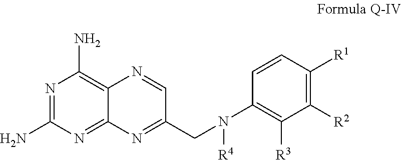

wherein
(i) R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —CH₂P(O)(OR⁵)₂), —OCH₂COOR⁵; —C(O)N(R⁶)S(O)₂R⁷, —C(H)═C(OH)C(O)OR⁵, —C(H)(NH₂)COOR⁵, —COOR⁵, C(O)N(Rₐ)(R_{b}) or —OCH₂C(O)N(Rₐ)(R_{b});
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
   in free, salt or prodrug form.

In still another embodiment of the first aspect, the invention provides a Compound of Formula I:

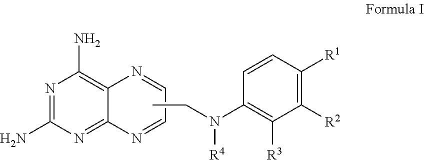

wherein
(vii) R¹, R² and R³ are as follows:
   (a) R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, or —C(H)═C(OH)C(O)OR⁵;
(b) R² and R³ are independently COOR⁵; or
(c) R² and R³ are independently —OH or —OCH₂COOR⁵ and R¹ is —COOR⁵;
(viii) R⁴ is a H or C₁₋₄alkyl (e.g., methyl);
(ix) R⁵ is a H or C₁₋₄alkyl (e.g., ethyl);
(x) R⁶ is H or C₁₋₄alkyl;
(xi) R⁷ is C₁₋₄alkyl (e.g., methyl);
   in free, salt or prodrug form.

In another embodiment, the invention provides a Compound of Formula II:

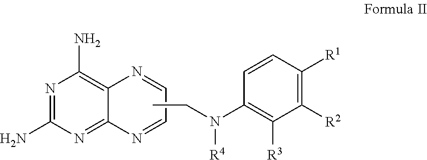

wherein
(i) R¹, R² and R³ are as follows:
   (a) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR¹)₂), —OCH₂COOR⁵; —C(O)N(R⁶)S(O)₂R⁷, or —C(H)═C(OH)C(O)OR⁵; or
   (b) R² and R³ are independently —OH or —OCH₂COOR⁵ and R¹ is —COOR^{S};
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl),
   in free, salt or prodrug form.

In another embodiment, the invention provides a Compound of Formula III:

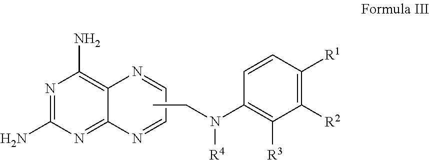

wherein
(vii) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, or —C(H)═C(OH)C(O)OR⁵;
(viii) R⁴ is a H or C₁₋₄alkyl (e.g., methyl);
(ix) R⁵ is a H or C₁₋₄alkyl (e.g., ethyl);
(x) R⁶ is H or C₁₋₄alkyl;
(xi) R⁷ is C₁₋₄alkyl (e.g., methyl);
   in free, salt or prodrug form.

In another embodiment, the invention provides a compound of Formula IV:

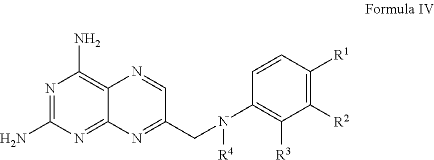

wherein
(ii) R¹, R² and R³ independently H, —SO₂CF₃, —SO₃H, —NH₂, —C(O)N(R)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵′, —C(H)(NH₂)COOR⁵ or —COOR⁵;
(vii) R⁴ is a H or C₁₋₄alkyl (e.g., methyl);
(viii) R⁵ is a H or C₁₋₄alkyl (e.g., ethyl);
(ix) R⁶ is H or C₁₋₄alkyl;
(x) R⁷ is C₁₋₄alkyl (e.g., methyl);
   in free, salt or prodrug form.

In a further embodiment, the invention provides a compound as follows:
1.1. a compound of Formula I, II or III, having the following configuration:

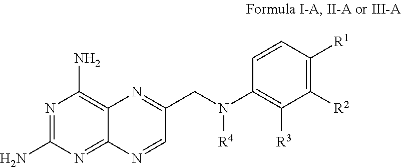

1.2. a compound of Formula I, II or III, having the following configuration:

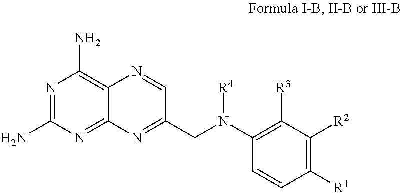

1.3. a compound of Formula I, II, III or IV, 1.1 or 1.2, wherein R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR¹)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷ or —C(H)═C(OH)C(O)OR⁵;
1.4. a compound of Formula I, II or III, or any of 1.1-1.3, wherein R¹, R² and R³ are independently H, —SO₂CF₃, —SO₃H, —CH₂C(O)COOR₅, —P(O)(OR⁵)₂ or —OCH₂COOR⁵;
1.5. a compound of Formula I, II, III or IV, or any of 1.1-1.3 wherein R¹, R² and R³ are independently H,
1.6. a compound of Formula I, II, III or IV, or any of 1.1-1.3 wherein R¹, R² and R³ are independently —SO₂CF₃;
1.7. a compound of Formula I, II, III or IV, or any of 1.1-1.3 wherein R¹, R² and R³ are independently —SO₃H,
1.8. a compound of Formula I, II, III or IV, or any of 1.1-1.3 wherein R¹, R² and R³ are independently —NH₂;
1.9. a compound of Formula I, II, III or IV, or any of 1.1-1.3 wherein R¹, R² and R³ are independently —C(O)N(R⁶)SO₃H;
1.10. a compound of Formula I, II, III or IV, or any of 1.1-1.3 wherein R¹, R² and R³ are independently —CH₂C(O)COOR₅;
1.11. a compound of Formula I, II, III or IV, or any of 1.1-1.3 wherein R¹, R² and R³ are independently —OH;
1.12. a compound of Formula I, II, III or IV, or any of 1.1-1.3 wherein R¹, R² and R³ are independently —C₁₋₄alkyl-COOR⁵ (e.g., —CH₂COOR⁵);
1.13. a compound of Formula I, II, III or IV, or any of 1.1-1.3 wherein R¹, R² and R³ are independently —P(O)(OR⁵)₂ (e.g., —P(O)(OCH₂CH₃)₂ or —P(O)(OF)₂);
1.14. a compound of Formula I, II, III or IV, or any of 1.1-1.3 wherein R¹, R² and R³ are independently —C₁₋₄alkyl-P(O)(OR⁵)₂ (e.g., —CH₂P(O)(OR⁵)₂, e.g., —CH₂P(O)(OH)₂);
1.15. a compound of Formula I, II, III or IV, or any of 1.1-1.3 wherein R¹, R² and R³ are independently —OCH₂COOR⁵;
1.16. a compound of Formula I, II, III or IV, or any of 1.1-1.3 wherein R¹, R² and R³ are independently —C(O)N(R⁶)S(O)₂R⁷;
1.17. a compound of Formula I, II, III or IV, or any of 1.1-1.3 wherein R¹, R² and R³ are independently —C(H)═C(OH)C(O)OR⁵;
1.18. a compound of Formula I, II or IV, or any of 1.1-1.3 wherein R² and R³ are independently —OH or —OCH₂COOR⁵ and R¹ is —COOR⁵;
1.19. a compound of Formula I, II or IV, or any of 1.1-1.3 wherein R² is —OH and R¹ is —COOR⁵;
1.20. a compound of Formula I, II or IV, or any of 1.1-1.3 wherein R² is —OCH₂COOR⁵ and R¹ is —COOR⁵;
1.21. a compound of Formula I, II or IV, or any of 1.1-1.3 wherein R² and R³ are independently COOR⁵;
1.22. a compound of Formula I, II, III or IV, or any of 1.1-1.3 wherein R¹, R² and R³ are independently H, —C₁₋₄alkyl-COOR⁵ (e.g., —CH₂COOR⁵), —C(H)═C(OH)C(O)OR⁵, —P(O)(OR⁵)₂, —C₁₋₄alkyl-P(O)(OR⁵)₂ (e.g., —CH₂P(O)(OR⁵)₂);
1.23. a compound of Formula I, II, III or IV, or any of 1.1-1.22, wherein R⁴ is H or C₁₋₄alkyl (e.g., methyl);
1.24. a compound of Formula I, II, III or IV, or any of 1.1-1.23, wherein R⁴ is H,
1.25. a compound of Formula I, II, III or IV, or any of 1.1-1.23, wherein R⁴ is C₁₋₄alkyl (e.g., methyl);
1.26. a compound of Formula I, II, III or IV, or any of 1.1-1.25, wherein R⁵ is H or C₁₋₄alkyl (e.g., ethyl);
1.27. a compound of Formula I, II, III or IV, or any of 1.1-1.26, wherein R₁ is H,
1.28. a compound of Formula I, II, III or IV, or any of 1.1-1.26, wherein R⁵ is C₁₋₄alkyl (e.g., ethyl);
1.29. a compound of Formula I, II, III or IV, or any of 1.1-1.28, wherein R⁶ is H or C₁₋₄alkyl;
1.30. a compound of Formula I, II, III or IV, or any of 1.1-1.28, wherein R⁶ is H,
1.31. a compound of Formula I, II, III or IV, or any of 1.1-1.28, wherein R⁶ is
1.32. a compound of Formula I, II, 111 or IV, or any of 1.1-1.31, wherein R⁷ is C₁₋₄alkyl (e.g., methyl);
1.33. a compound of Formula I, II, III or IV or any of 1.1-1.32 selected from any of the following:

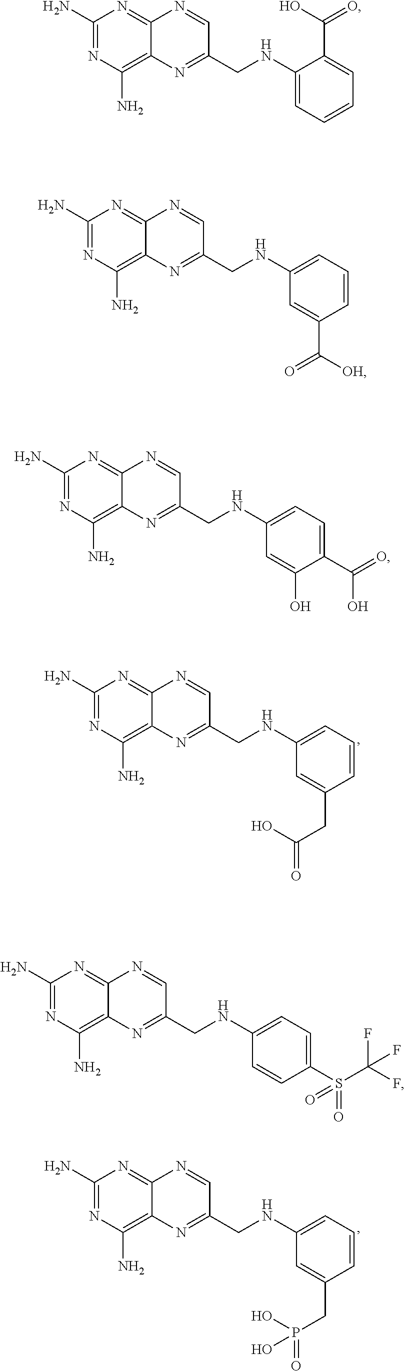

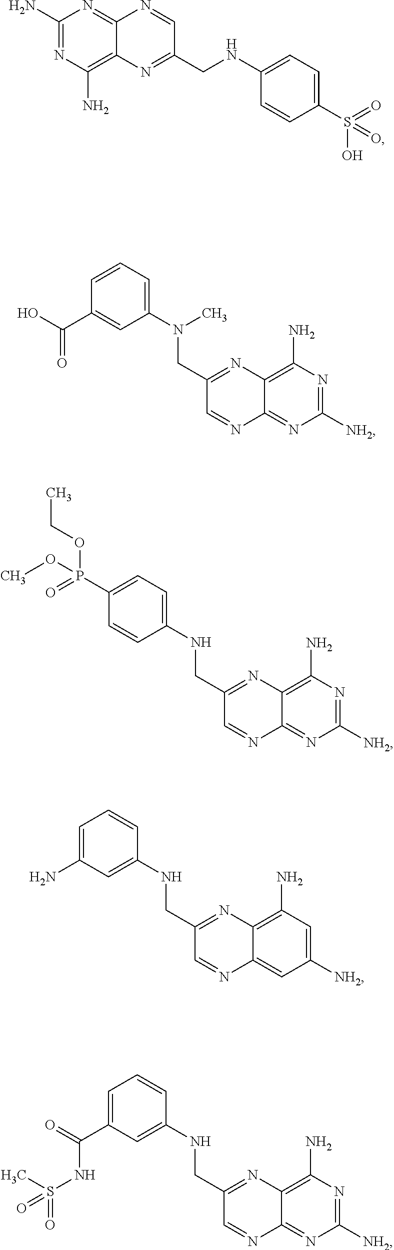

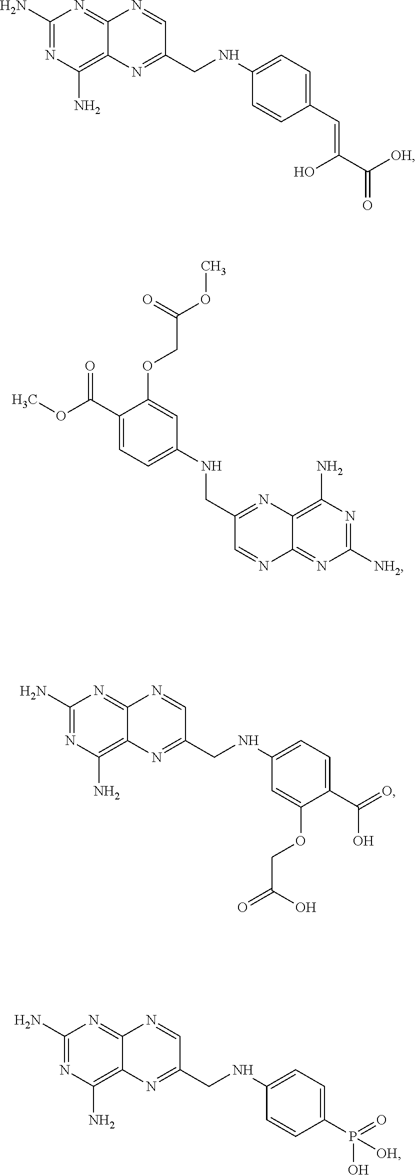

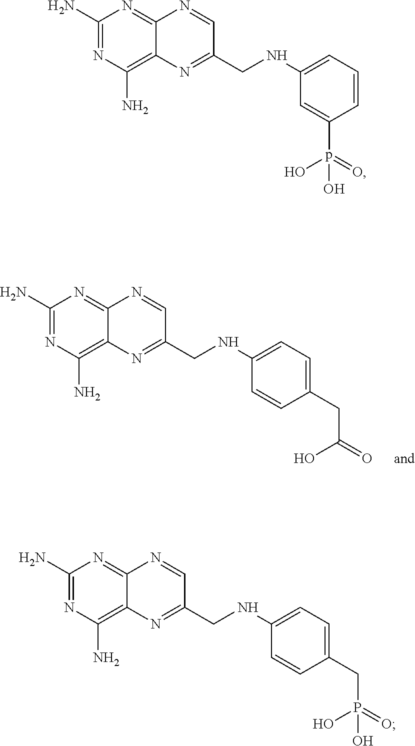

1.34. a compound of Formula II or III or any of 1.1-1.33 selected from any of the following:

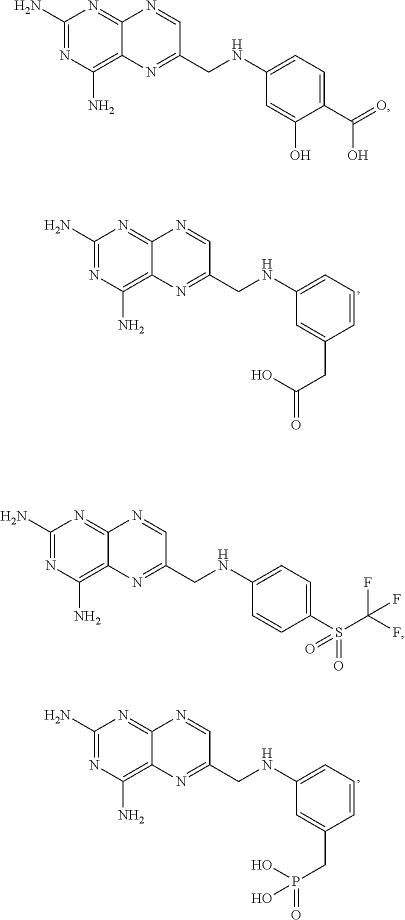

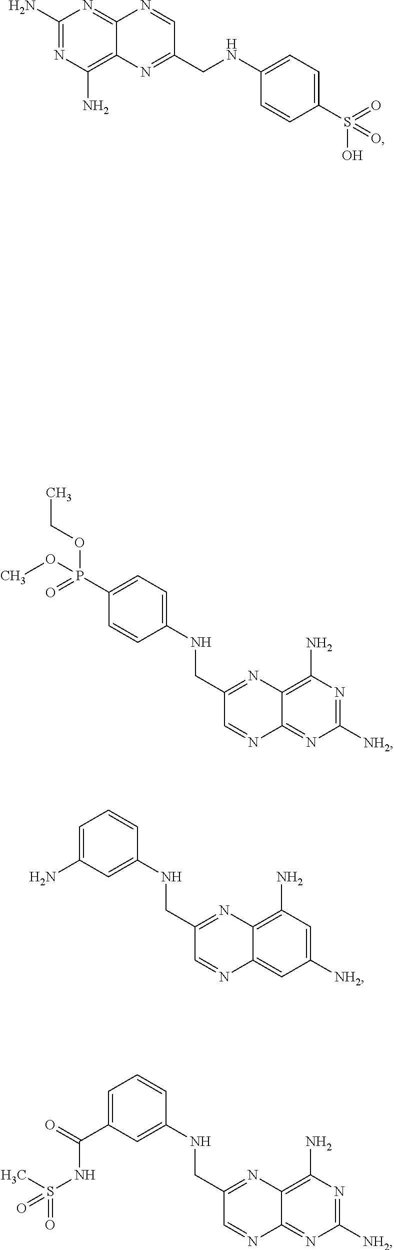

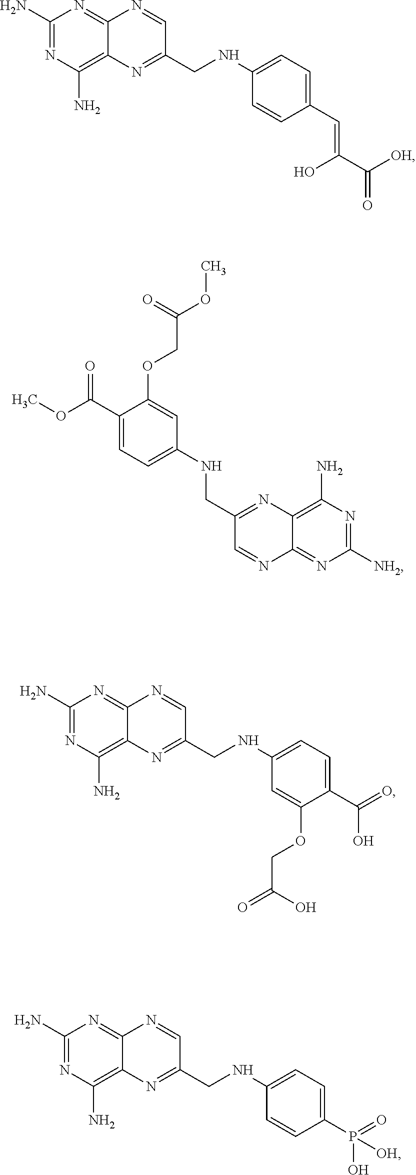

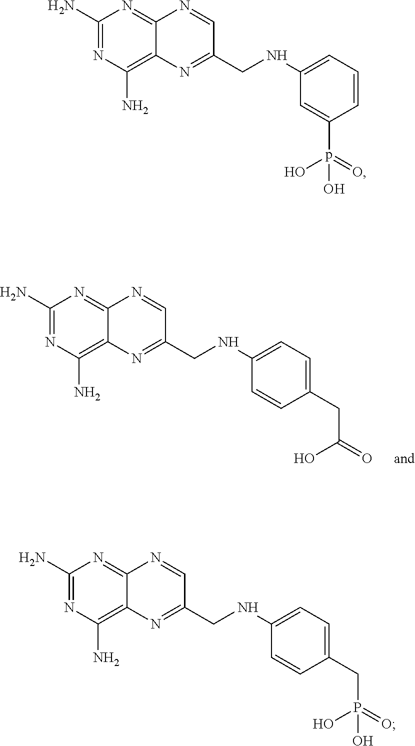

1.35. a compound of Formula III or any of 1.1-1.33 selected from any of the following:

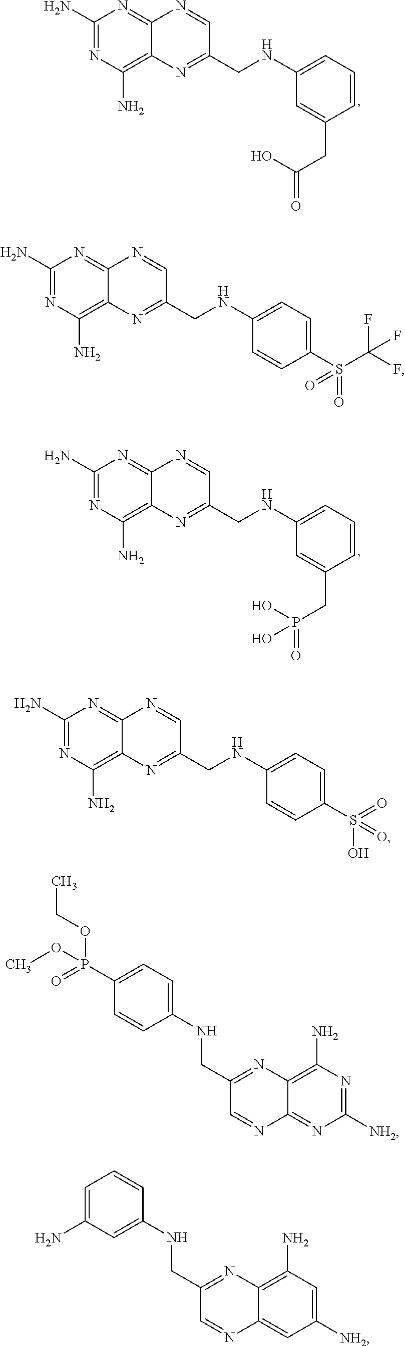

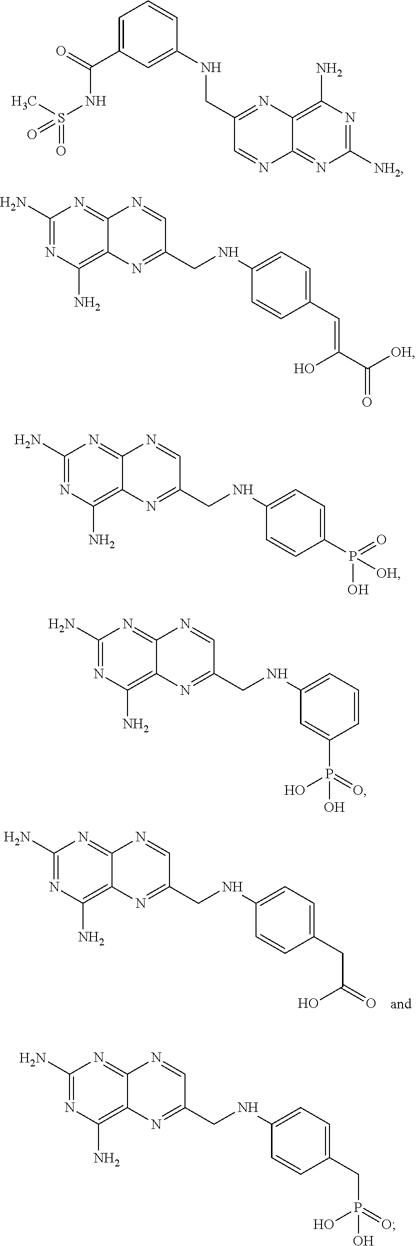

1.36. a compound of Formula I, II or III, or any of 1.1-1.32 selected from any of the following:

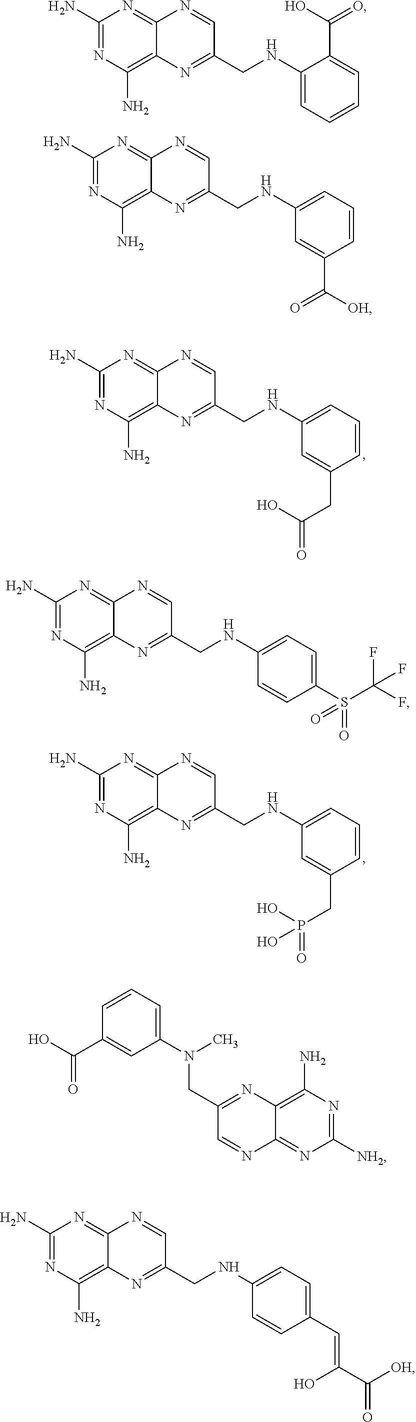

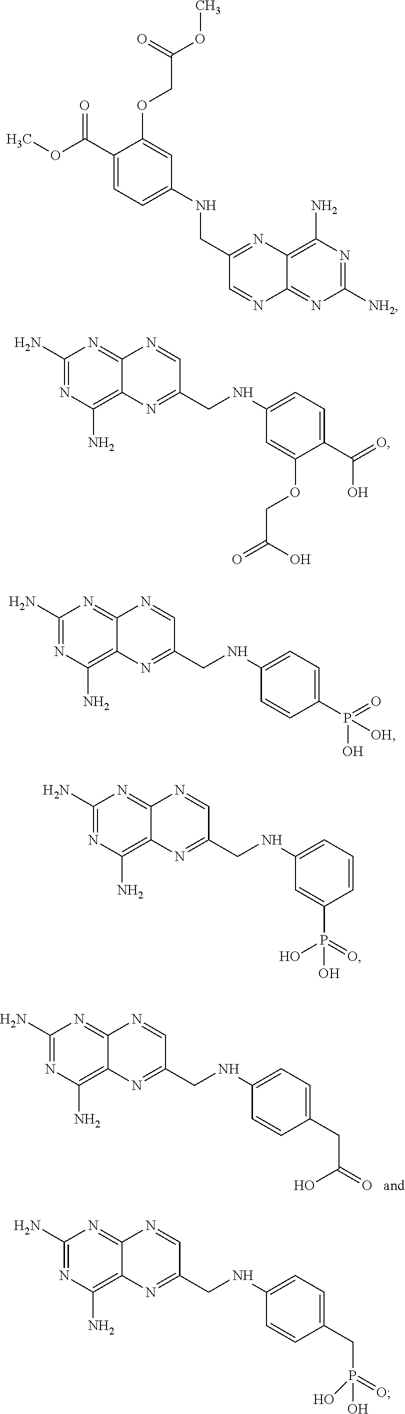

1.37. a compound of Formula I, II or III or any of 1.1-1.32 selected from any of the following:

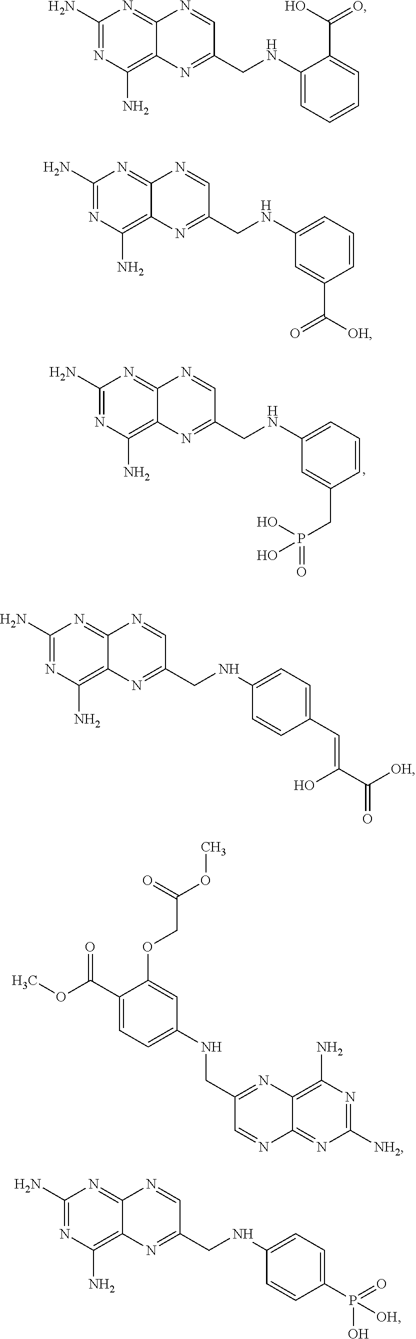

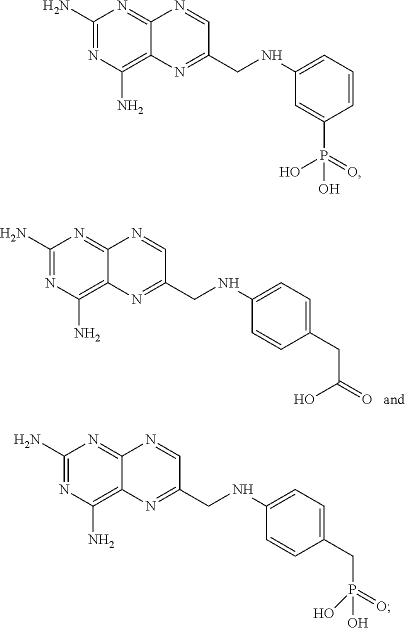

1.38. a compound of Formula I, II or III or any of 1.1-1.33 selected from any of the following:

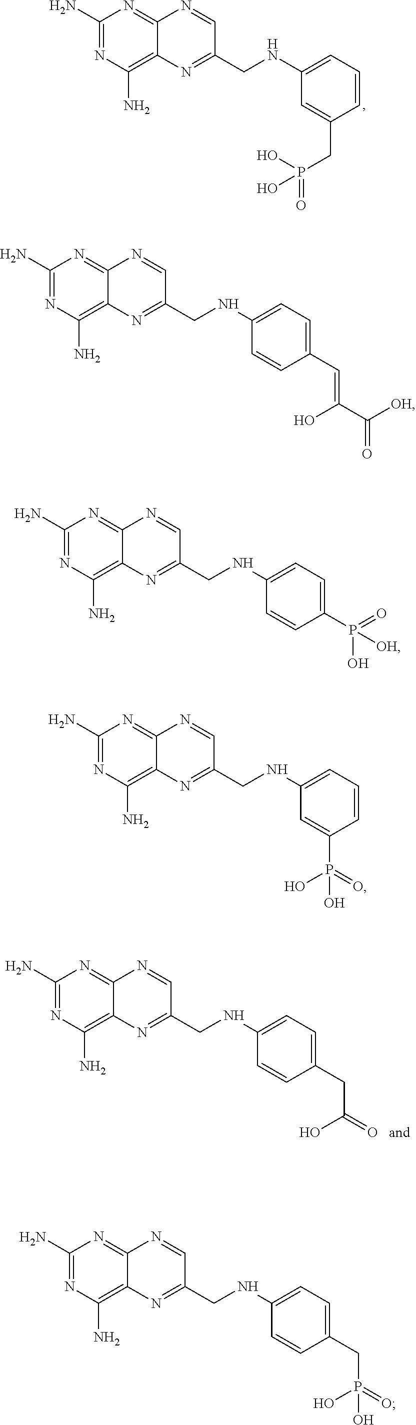

1.39. a compound of Formula I or any of 1.1-1.33 selected from any of the following:

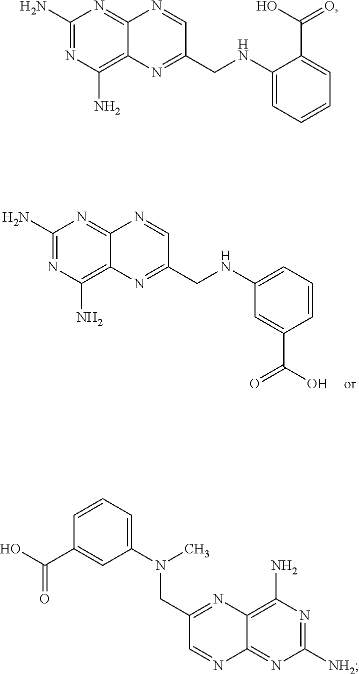

1.40. a compound of Formula I, II or III or any of 1.1-1.33 selected from any of the following:

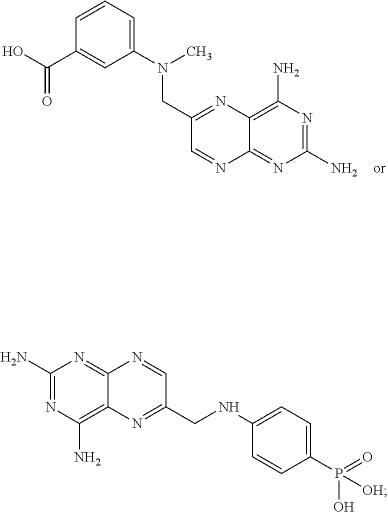

1.41. a compound of Formula IV, or any of 1.2-1.33 wherein R¹, R² and R³ are independently —C(H)(NH₂)COOR⁵;
1.42. a compound of Formula IV, or any of 1.2-1.33 wherein R¹, R² and R³ are independently —COOR⁵ (e.g., —COOH);
1.43. a compound of Formula IV, or any of 1.2-1.33, wherein the compound is:

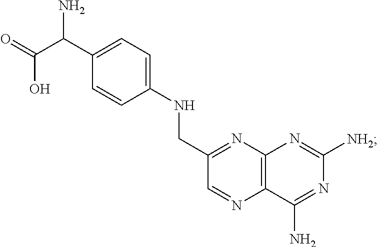

1.44. a compound of Formula I, II, III or IV or any of 1.1-1.43, wherein said compound has a minimum inhibitory concentration (MIC) of less than 130 μg/mL, preferably less than 100 μg/mL, more preferably less than 50 μg/mL, still more preferably less than 25 μg/mL, most preferably, less than 10 μg/mL in an assay, for example, as described in Example 1.
   in free, salt or prodrug form.

In another embodiment, the invention provides the following formulae:
2.1 a compound of Formula Q-I, Q-II, Q-III or Q-IV, as hereinbefore described, wherein the substituents are described in any of formulae 1.1-1.44;
2.2 a compound of Formula Q-I, wherein:
   R¹, R² and R³ independently H, —SO₂CF₃, —SO₃H, —NH₂, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
   R² and R³ are independently COOR⁵; or
   R² and R³ are independently —OH or —OCH₂COOR⁵ and R¹ is —COOR⁵
   and the remaining substituents are described in any of formulae 1.1-1.2, 1.4-1.8, 1.10-1.28 and 1.32-1.44;
2.3 a compound of Formula Q-II, wherein:
   R¹, R² and R³ are independently H, —SO₂CF₃, —SO₃H, —NH₂, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵ or
   R² and R³ are independently —OH or —OCH₂COOR⁵ and R¹ is —COOR⁵;
   and the remaining substituents are described in any of formulae 1.1-1.2, 1.4-1.8, 1.10-1.28 and 1.32-1.44;
2.4 a compound of Formula Q-III, wherein:
   R¹, R² and R³ are independently H, —SO₂CF₃, —SO₃H, —NH₂, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
   and the remaining substituents are described in any of formulae 1.1-1.2, 1.4-1.8, 1.10-1.28 and 1.32-1.44;
2.5 a compound of Formula Q-IV, wherein:
   R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(H)(NH₂)COOR⁵, —COOR⁵, C(O)N(Rₐ)(R_{b}) or —OCH₂C(O)N(Rₐ)(R_{b});
   and the remaining substituents are described in any of formulae 1.1-1.2, 1.4-1.8, 1.10-1.28 and 1.32-1.44;
2.6 a compound of Formula Q-I, Q-II, Q-III or Q-IV, or any of formulae 2.1-2.5, wherein R¹, R² and R³ are independently —CH₂CH₂COOR⁵;
2.7 a compound of Formula Q-I, Q-II, Q-III or Q-IV, or any of formulae 2.1-2.6, selected from any of the following:

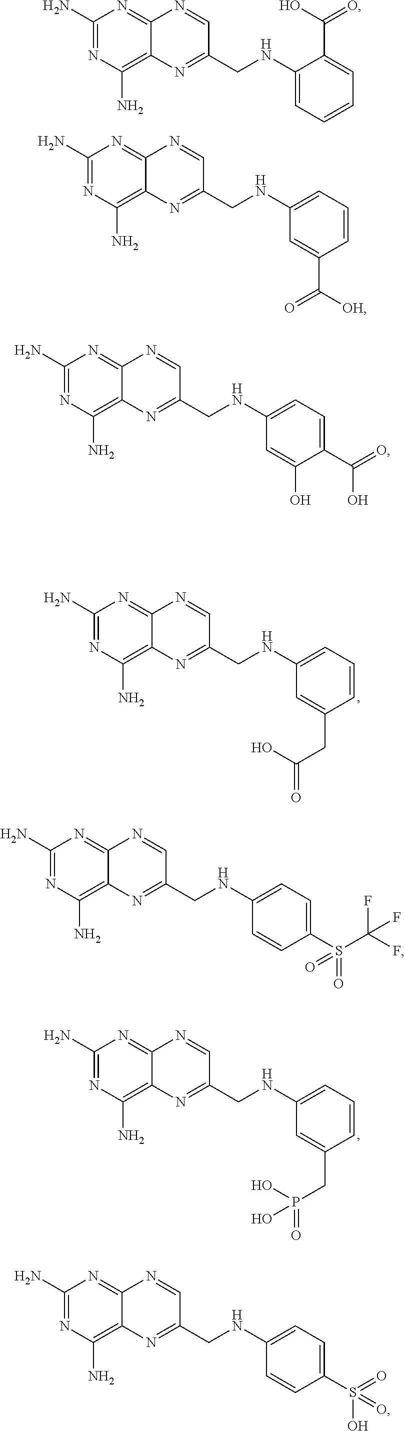

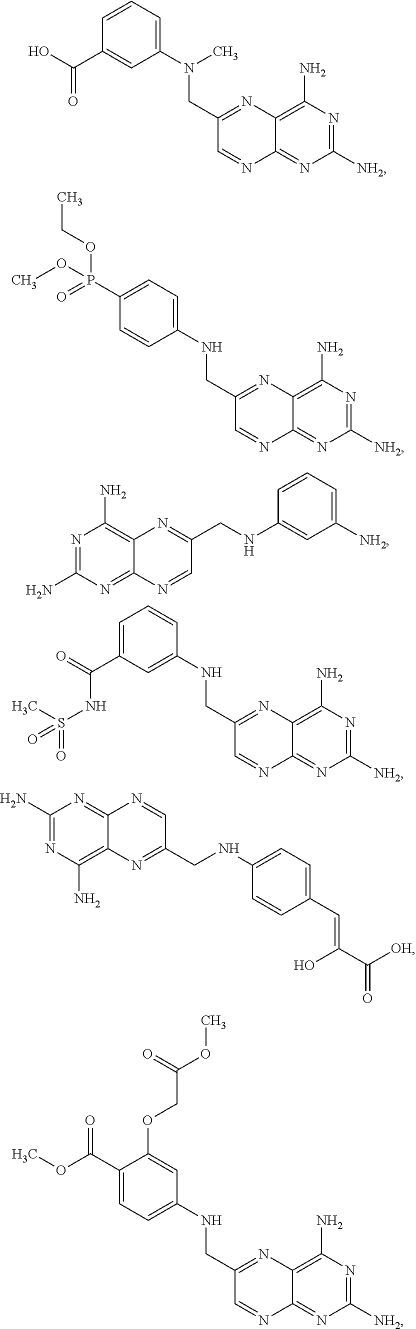

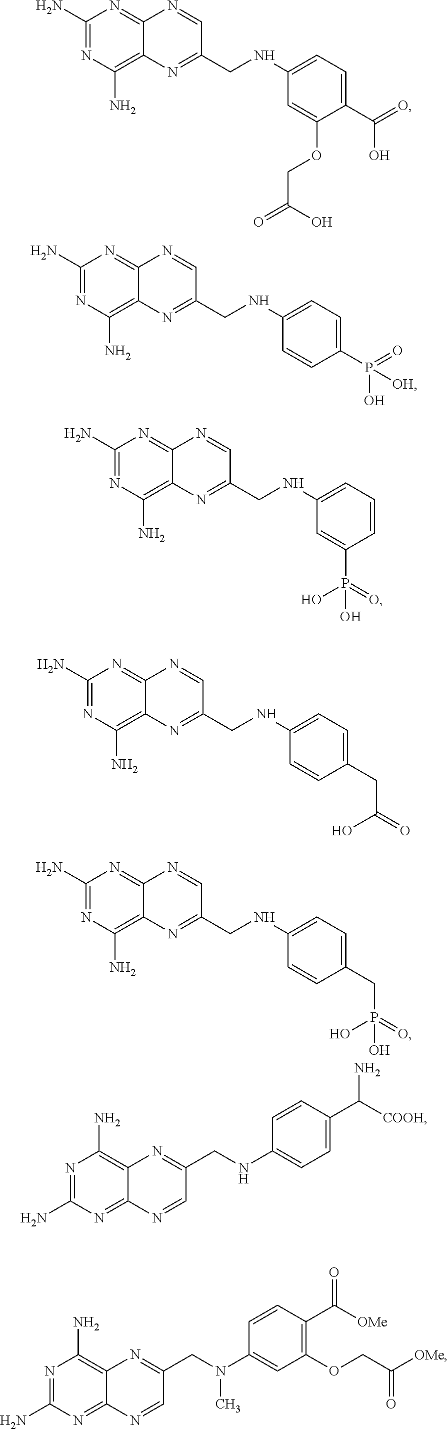

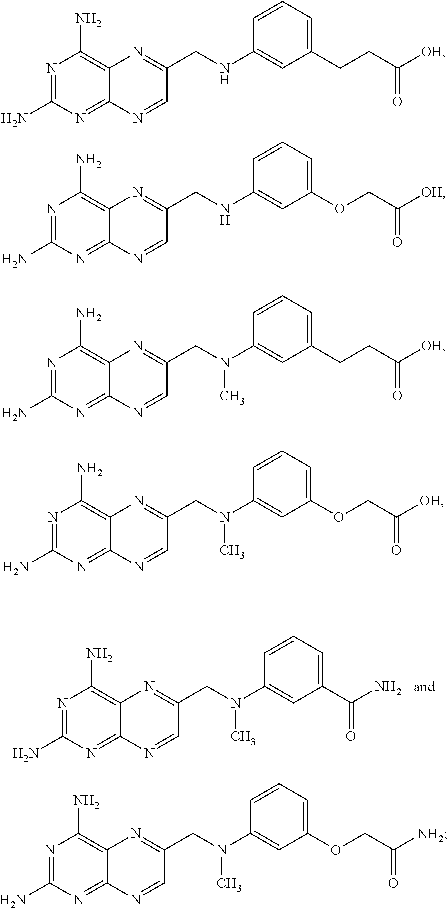

in free, salt or prodrug form.

In still another embodiment, the invention provides a compound of Formula V:

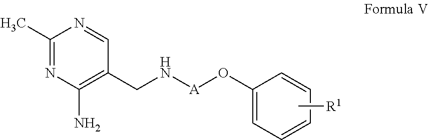

wherein:
A is a heteroaryl, e.g.,

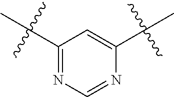

R¹ is C₁₋₈alkyl-COOR² (e.g, —CH₂COOR²)
R² is H or C₁₋₄alkyl (e.g., methyl);
   in free, salt or prodrug form.

In a further embodiment, the compound of Formula V is selected from:

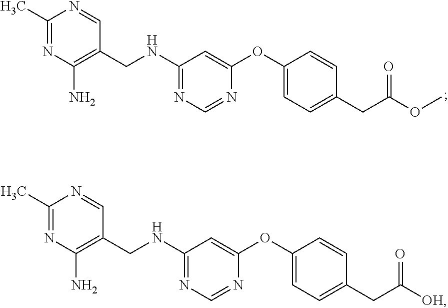

in free, salt, or prodrug form (Formula 5.1).

In another embodiment, the invention provides a compound of Formula VI:

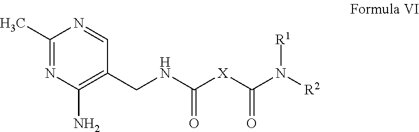

wherein:
X is a C₁₋₄alkyl, e.g., ethyl;
R¹ and R² are independently H or C₁₋₈alkyl-P(O)(OR³)(OR⁴), e.g, —CH₂CH₂P(O)(OR³)(OR⁴);
R³ and R⁴ are independently H, C₁₋₄alkyl (e.g., ethyl) or C₁₋₄alkyl-OC(O)R⁵ (e.g., —CH₂CH₂OC(O)R⁵;
R⁵ is C₁₋₄alkyl (e.g., t-butyl),
   in free, salt or prodrug form.

In a further embodiment, the compound of Formula VI is selected from:

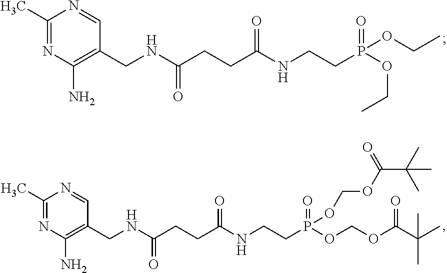

in free, salt or prodrug form (Formula 6.1).

The present invention claims a compound of any of formulae I-IV, e.g., any of formulae 1.1-1.44, formulae Q-1 though Q-IV, e.g., any of 2.1-2.7, or formula V or VI, e.g., formula 5.1 or 6.1, in free, salt or prodrug form.

The Compounds of the Invention as hereinbefore described, a Compound of any of formulae I-IV, e.g., any of formulae 1.1-1.44, formulae Q-I though Q-IV, e.g., any of 2.1-2.7, or formula V or VI, e.g., formula 5.1 or 6.1, are useful as anti-infectives, e.g., anti-bacterial or anti-fungal. These compounds may act via a number of mechanisms, e.g., by inhibiting synthesis of bacterial cell walls, by acting directly on cell membrane of the microorganism to increase permeability and leakage of intracellular compounds, by disrupting function of various ribosomal subunits to reversibly inhibit protein synthesis, by binding to various ribosomal subunit and alter protein synthesis, by affecting bacterial nucleic acid metabolism or by blocking essential enzymes of folate metabolism, such as dihyrdrofolate reductase or dihydropteroate synthase. Without intending to be bound by any particular theory, it is believed that, in a particular instance, certain pteridine derivatives of the invention, e.g., certain compounds of formula I, II, III or IV as hereinbefore described, e.g., any of formulae 1.1-1.44, any of formulae Q-I though Q-IV, e.g., 2.1-2.7, or formula V, e.g., formula 5.1, preferably any of formulae 1.37, 1.38, 1.43, or 5.1, in free, salt or prodrug form, also targets thiamine pyrophosphate riboswitch, e.g., binds TPP riboswitch with an IC₅₀ value of less than 20 μM, preferably less than 10 μM, most preferably less than 1 μM in a binding assay, for example, as described in Example 2. As such, the invention also provides various Compounds of formula I, e.g., any of formulae 1.1-1.44, Q-I though Q-IV, e.g., 2.1-2.7, or formula V, e.g., 5.1, preferably any of formulae 1.37, 1.38, 1.43, or 5.1, in free, salt or prodrug form, as TPP riboswitch ligand.

In another aspect, the invention provides a pharmaceutical composition comprising a Compound of Formula I, II, III or IV, as herein before described, e.g., any of formulae 1.1-1.44, any of formulae Q-I though Q-IV, e.g., any of formulae 2.1-2.7, or formula V or VI, e.g., formula 5.1 or 6.1, in free, pharmaceutically acceptable salt or prodrug form, in admixture with a pharmaceutically acceptable diluent or carrier.

In still another aspect, the invention provides a method for the treatment or prophylaxis of an infection (Method 1) comprising administering to a subject in need thereof an effective amount of a compound of formula I, II, III or IV as hereinbefore described, e.g., any of formulae 1.1-1.44, Q-I though Q-IV, e.g., any of 2.1-2.7, or formula V or VI, e.g., formula 5.1 or 6.1, in free, pharmaceutically acceptable salt or prodrug form, or a pharmaceutical composition comprising the same.

In a further embodiment, Method 1 as hereinbefore described, is useful for the treatment or prophylaxis of a Gram-positive or Gram-negative bacterial infection (Method I-A). In another specific embodiment, Method 1 is useful for treating a bacterial infection including, but not limited to an infection by one or more of the following bacteria: Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi (Method 1-B). In another embodiment, the Method 1 is useful for the treatment or prophylaxis of an infection by one or more of the following bacteria: Staphylococcus aureus, Staphylococcus epidermidis, Bacillus subtilis, Enterococcus faecalis, Streptococcus pneumoniae, Streptococcus pyogenes, Escherichia coli. Pseudomonas aeruginosa, Klebsiella pneumoniae, Haemophilus influenzae and Acinetobacter baumannii (Method 1-B′). In yet another embodiment, the Method 1 is useful for the treatment or prophylaxis of an infection by one or more of the following bacteria: Staphylococcus aureus, Staphylococcus epidermidis, Enterococcus faecalis, Streptococcus pneumoniae, Escherichia coli, Pseudomonas aeruginosa, Klebsiella pneumonia and Haemophilus influenza. In a particular embodiment, Method 1 is useful for the treatment or prophylaxis of an infection by one or more of the following bacteria Staphylococcus aureus, Streptococcus pneumoniae and Streptococcus pyogenes (Method 1-B″). In a particular embodiment, Method 1 is useful for the treatment or prophylaxis of a Staphylococcus aureus infection (Method 1-C).

In a further embodiment, Method 1 as hereinbefore described is useful for the treatment or prophylaxis of a disease, infection or condition selected from a group consisting of anthrax, staphylococcal scalded skin syndrome (staph infections), lyme disease, pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound of formula I, II, III or IV as hereinbefore described, e.g., any of formulae 1.1-1.44, any of formulae Q-I though Q-IV, e.g., any of 2.1-2.7, or formula V or VI, e.g., formula 5.1 or 6.1, in free, pharmaceutically acceptable salt or prodrug form, or a pharmaceutical composition comprising the same, (Method 1-D).

Without wishing to be bound to any particular theory, it is believed that particular Compounds of the Invention, various compounds of Formula I, II, III, or IV, e.g., of formulae 1.1-1.44, of formulae Q-1 though Q-IV, e.g., of 2.1-2.7, or formula V or VI, e.g., formula 5.1 or 6.1, preferably formula 1.37, 1.38, 1.43 or 5.1, in free, pharmaceutically acceptable salt or prodrug form, also target thiamine pyrophosphate riboswitch, and therefore provide methods of treating a bacterial infection via a novel mechanism, e.g., by utilizing riboswitch-ligand binding to alter gene expression, thereby affecting downstream thiamine biosynthesis. As such, these compounds are effective in treating an infection wherein traditional antibiotics are rendered ineffective due to drug resistance. Therefore, in a particular embodiment, the invention provides Method 1 or any of Methods 1-A to 1-D as hereinbefore described wherein the compound is a compound of any of formulae 1.37, 1.38, 1.43 or 5.1, in free, pharmaceutically acceptable salt or prodrug form, and wherein the infection is by an infectious agent which is resistant to a drug that is not a riboswitch ligand (Method 1-E). In a further embodiment, the infection is resistant to one or more drugs selected from a group consisting of a penicillin, vancomycin, cephalosporin and methicillin. In a particular embodiment, the infection is a methicillin-resistant Staphylococcus aureus infection.

In another aspect, the invention provides use of a Compound of Formula I, II, III or IV e.g., any of formulae 1.1-1.44, any of formulae Q-I though Q-IV, e.g., any of 2.1-2.7, or formula V or VI, e.g., formula 5.1 or 6.1, in free, pharmaceutically acceptable salt or prodrug form, in the manufacture of a medicament for the treatment or prophylaxis of an infection.

In a particular embodiment, the invention provides use as hereinbefore described, wherein the infection is a Gram-positive or Gram-negative infection. In still another specific embodiment, the infection is an infection of one or more bacteria selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio chlierae, Enterococcus faecalis, Yersinia pestis, Bacillus subtilis, Streptococcus pyogenes and Borrelia burgdorferi. In still another specific embodiment, the infection is an infection of one or more bacteria selected from a group consisting of Staphylococcus aureus, Staphylococcus epidermidis, Enterococcus faecalis, Streptococcus pneumoniae, Escherichia coli, Pseudomonas aeruginosa, Klebsiella pneumonia and Haemophilus influenzae In a preferred embodiment, the invention provides use as hereinbefore described wherein the infection is by one or more of the following bacteria: Staphylococcus aureus, Streptococcus pneumoniae and Streptococcus pyogenes. In a further embodiment, the invention provides use as herein described in the manufacture of a medicament for the treatment or prophylaxis of a condition, disease or infection selected from anthrax, staphylococcal scalded skin syndrome (staph infections), lyme disease, pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis.

In yet another embodiment, the invention provides use of various Compounds of Formula I, II, III, or IV, e.g., any of formulae 1.1-1.44, any of formulae Q-I though Q-IV, e.g., any of 2.1-2.7, or formula V, e.g., formula 5.1, preferably formula 1.37, 1.38, 1.43, or 5.1, in free, pharmaceutically acceptable salt or prodrug form, as hereinbefore described in Methods I, wherein said infection is resistant to a drug that is not a riboswitch ligand. In another further embodiment, the infection is resistant to one or more drugs selected from a group consisting of penicillin, vancomycin, cephalosporin and methicillin. In a particular embodiment, the infection is a methicillin-resistant Staphylococcus aureus infection.

In yet another embodiment, the invention provides use of various Compounds of Formula I, II, III or IV e.g., of formulae 1.1-1.44, of formulae Q-1 though Q-IV, e.g., of 2.1-2.7, or formula V, e.g., formula 5.1, preferably formula 1.37, 1.38, 1.43 or 5.1, in free, salt or prodrug form, in the manufacture of a medicament for the treatment or prophylaxis of a fungal infection.

In yet another embodiment, the invention provides a method for the treatment of an infection in a plant comprising administering to such plant comprising administering to such plant an effective amount of a Compound of Formula I, II, III or IV e.g., any of formulae 1.1-1.44, any of formulae Q-I though Q-IV, e.g., any of 2.1-2.7, or formula V, e.g., formula 5.1, preferably, any of formula 1.37, 1.38, 1.43 or 5.1, in free, salt or prodrug form, as hereinbefore described. In a particular embodiment, the infection is a bacterial or fungal infection in a plant.

The invention also provides a pharmaceutical composition comprising a Compound of formula I, II, III or IV e.g., any of 1.1-1.44, any of formulae Q-I though Q-IV, e.g., any of 2.1-2.7, or formula V or VI, e.g., formula 5.1 or 6.1 as hereinbefore described for use in the treatment of any disease or condition as hereinbefore described.

### DETAILED DESCRIPTION OF THE INVENTION

The term “riboswitch” or “riboswitches” is an art recognized term and refers to an mRNA which comprises a natural aptamer that binds target metabolite and an expression platform which changes in the RNA structure to regulate genes.

The term “TPP riboswitch” refers to riboswitch that binds to thiamine pyrophosphate or TPP-dependent protein effector.

“TPP riboswitch ligand” refers to any compound which binds to the TPP riboswitch, e.g., via the highly conserved TPP-binding aptamer in the 5′-untranslated regions of the mRNA's. Without wishing to be bound by any particular theory, it is believed the binding of the ligand to its riboswitch induces a conformational change in the bacterial mRNA such that the expression of the ORF is repressed, for example, such that the expression of enzymes responsible for thiamine biosynthesis is repressed. This is achieved by inducing the mRNA to form a terminator hairpin that halts RNA synthesis before the ORF can be synthesized or a hairpin that sequesters the Shine-Dalgarno sequence and prevents the ribosome from binding to the mRNA so as to translate the ORF. Examples of TPP riboswitch ligands include but are not limited to various compounds of Formula I, II, III, or IV, various compounds of formulae Q-I though Q-IV, e.g., any of formulae 1.37, 1.38, 1.43, or 5.1, in free, or salt form.

The term “infection” encompasses any infection by bacteria and/or fungi.

In a particular embodiment, the term “infection” refers to a bacterial infection. In another embodiment, the infection is a Gram-positive or Gram-negative infection. In still another embodiment, the infection is an infection by one or more bacteria selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi. In yet another embodiment, the infection is an infection by one or more bacteria selected from a group consisting of Staphylococcus aureus, Staphylococcus epidermidis, Enterococcus faecalis, Streptococcus pneumoniae, Escherichia coli, Pseudomonas aeruginosa, Klebsiella pneumonia and Haemophilus influenza. In a particular embodiment, the infection is an infection by one or more bacteria selected from Staphylococcus aureus, Streptococcus pneumoniae and Streptococcus pyogenes. In a further embodiment, the infection is a Staphylococcus aureus infection. In a particular embodiment, the infection is an infection which is resistant to a drug which is not a riboswitch ligand. In a further aspect of this particular embodiment, the infection is an infection which is resistant to one or more drugs selected from a group consisting of penicillin, vancomycin, cephalosporin and methicillin. In a particular embodiment, the infection is a methicillin-resistant Staphylococcus aureus (MRSA) infection.

In other aspect, the term “infection” refers to a fungal infection. Examples of a fungal infection include but are not limited to infection by Microsporum, Trichophyton, Epidermophyton, Tinea (e.g., tinea versicolor, tinea pedis, tinea corporis), Histoplasma capsulatum, Coccidioides immitis, Blastomyces dermatidis, Candida (e.g., Candida albicans), Aspergillus, fumigatus and Sporothrix schenckii fungi. Examples of conditions caused by a fungal infection include, but are not limited to mycoses such as superficial, cutaneous, subcutaneous or systemic mycosis, e.g., coccidioidomycosis, histoplasmosis, blastomycosis, candidiasis (e.g., yeast infection or moniliasis), sporotrichosis and ringworm (e.g., athlete's foot, jock itch, scalp ringworm, nail ringworm, body ringworm, beard ringworm).

The term “bacteria” or “bacterial” include, but are not limited to Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi. In a particular embodiment, the term “bacteria” or “bacterial” include, but are not limited Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In still another embodiment, the term “bacteria” or “bacterial” refers to Staphylococcus aureus, Staphylococcus epidermidis, Enterococcus faecalis, Streptococcus pneumoniae, Escherichia coli, Pseudomonas aeruginosa, Klebsiella pneumonia and Haemophilus influenza. In a preferred embodiment, the bacteria is selected from: Staphylococcus aureus, Streptococcus pneumoniae and Streptococcus pyogenes.

If not otherwise specified or clear from context, the following terms as used herein have the following meetings:
(i) “Alkyl” as used herein is a saturated or unsaturated hydrocarbon moiety, preferably saturated, e.g., one to eight carbon atoms in length, which may be linear or branched (e.g., n-butyl or tert-butyl), and may be optionally substituted, e.g., mono-, di-, or tri-substituted on any one of the carbon atom, e.g., with alkyl (e.g., methyl), alkoxy, halogen (e.g., chloro or fluoro), haloalkyl (e.g., trifluoromethyl), hydroxy, and carboxy. For example, “C₁-C₈ alkyl” denotes alkyl having 1 to 8 carbon atoms. Examples of alkyl include, but are not limited to, methyl, ethyl, n-propyl, i-propyl, n-butyl, i-butyl, sec-butyl, t-butyl, 3-methylpentyl, 4-methylpentyl, n-pentyl, n-hexyl and n-heptyl.

Compounds of the Invention (e.g., Compounds of Formula I, II, III, or IV, e.g., any of formulae 1.1-1.44, Q-I though Q-IV, e.g., 2.1-2.7, or formula V or VI, e.g., 5.1 or 6.1) may exist in free or salt form, e.g., as acid addition salts. An acid-addition salt of a compound of the invention which is sufficiently basic, for example, an acid-addition salt with, for example, an inorganic or organic acid, for example hydrochloric, hydrobromic, sulphuric, phosphoric, acid acetic, trifluoroacetic, citric, maleic acid, toluene sulfonic, propionic, succinic, glycolic, stearic, lactic, malic, tartaric, citric, ascorbic, pamoic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. In addition a salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation. In this specification, unless otherwise indicated, language such as Compounds of the Invention is to be understood as embracing such Compounds in any form, for example free base or acid addition salt form, or where the compounds contain acidic substituents, in free acid or base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention or their pharmaceutically acceptable salts, are therefore also included.

Compounds of the Invention may in some cases also exist in prodrug form. The term “prodrug” is an art recognized term and refers to a drug precursor prior to administration, but generate or release the active metabolite in vivo following administration, via some chemical or physiological process. For example, when the Compounds of the Invention contain a carboxy or phosphonate substituent, these substituents may be esterified to form physiologically hydrolysable and acceptable esters (e.g., carboxylic acid esters or phosphonate esters, e.g., —C(O)OR⁵, —P(O)(OR⁵)(OR⁵). As used herein, “physiologically hydrolysable and acceptable esters” means esters of Compounds of the Present Invention which are hydrolysable under physiological conditions to yield acids, e.g., carboxylic acid or phosphonic acid (in the case of Compounds of the Invention which have carboxy or phosphonate substituents) on the one hand and HOR⁵ or HOR⁵ on the other hand, which are themselves physiologically tolerable at doses to be administered. Similarly, wherein the compounds of the invention contain an amine group, prodrug of such amine, for example, amino acid, carbamic acid ester, or amide prodrugs, may also exist wherein the prodrug is cleaved to release the active amine metabolite in vivo following administration. Further details of amine prodrugs may be found in Jeffrey P. Krise and Reza Oliyai, Biotechnology: Pharmaceutical Aspects, Prodrugs, Volume 5, Part 3, pages 801-831, the contents of which are herein incorporated by reference in their entirety. As will be appreciated, the term thus embraces conventional pharmaceutical prodrug forms.

### Methods of Making Compounds of the Invention

The compounds of the Formula I, II, III or IV, Q-I through Q-IV, or Formula V or VI, in free or salt form may be made using the methods as described and exemplified herein and by methods similar thereto and by methods known in the chemical art. Such methods include, but not limited to, those described below. In the description of the synthetic methods described herein, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. Therefore, at times, the reaction may require to be run at elevated temperature or for a longer or shorter period of time. It is understood by one skilled in the art of organic synthesis that functionality present on various portions of the molecule must be compatible with the reagents and reactions proposed. If not commercially available, starting materials for these processes may be made by procedures, which are selected from the chemical art using techniques which are similar or analogous to the synthesis of known compounds. All references cited herein are hereby incorporated in their entirety by reference.

The synthetic methods for the Compounds of the Present Invention are illustrated below. The significances for the R groups are as set forth above for Formula I, II, III or IV, or Formula Q-I through Q-IV unless otherwise indicated.

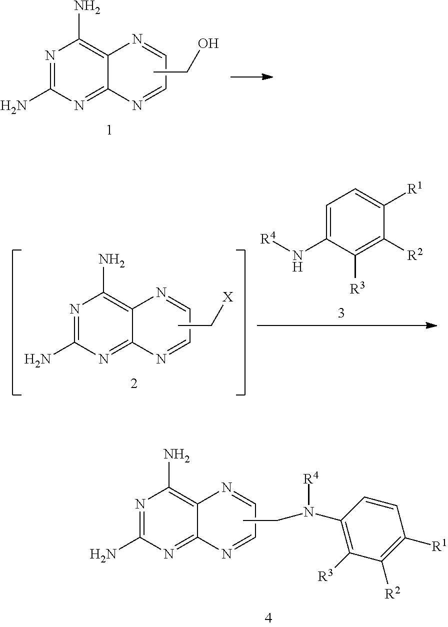

The Compounds of the Invention may be prepared by reacting, for example, 2,4-diaminopteridin-6-yl-methanol (1) to a 2,4-diaminopteridin-6-yl-methyl halide, 2, (e.g., 6-(bromomethyl)-pteridine-2,4-diamine) by reacting with, for example, SOCl₂, PCl_{S}, PCl_{S}, POCl₃, PBr₃, Ph₃P/Br₂, Ph₃P/Cl₂ or HX (e.g., HCl, HBr or HI). The 2,4-diaminopteridin-6-yl-methyl halide, 2, is then coupled with aniline 3 optionally in the presence of a base, e.g., potassium carbonate, sodium carbonate, sodium bicarbonate, triethyl amine, sodium hydride, barium oxide, or the like, to yield the Compound of the Invention, 4.

### Methods of Using Compounds of the Invention

The Compounds of the Invention are useful for the treatment of an infection, particularly an infection by bacteria including but not limited to Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi. In a particular embodiment, the Compounds of the Invention are useful for the treatment of an infection, particularly an infection by bacteria including but not limited to Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another embodiment, the Compounds of the Invention are useful for the treatment of an infection, particularly an infection by one or more of the following bacteria: Staphylococcus aureus, Streptococcus pneumoniae and Streptococcus pyogenes.

The invention therefore provides methods of treatment of any one or more of the following conditions: anthrax infection, staphylococcal scalded skin syndrome (staph infections), lyme disease, pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis; comprising administering an effective amount of a Compound of Formula I, II, III or IV, e.g., any of formulae 1.1-1.44, Formula Q-I through Q-IV, e.g., any of 2.1-2.7, or Formula V or VI, e.g., 5.1 or 6.1, in free, pharmaceutically acceptable salt or prodrug form, to a human or animal subject in need thereof.

The words “treatment” and “treating” are to be understood accordingly as embracing prophylaxis and treatment or amelioration of symptoms of disease as well as treatment of the cause of the disease.

The term “subject” as used herein encompasses human or non-human (e.g., animal) and/or plant.

Dosages employed in practicing the present invention will of course vary depending, e.g. on the particular disease or condition to be treated, the particular Compound of the Invention used, the mode of administration, and the therapy desired. Administration of a therapeutically active amount of the therapeutic compositions is defined as an amount effective, at dosages and for periods of time necessary to achieve the desired result. For example, a therapeutically effective amount of a Compound of the Present Invention reactive with at least a portion of TPP riboswitch may vary according to factors such as the disease state, age, sex, and weight of the individual, and the ability of the compound to elicit a desired response in the individual. Dosage regiment may be adjusted to provide the optimum therapeutic response. For example, several divided doses may be administered daily or the dose may be proportionally reduced as indicated by the exigencies of the therapeutic situation.

Pharmaceutical compositions comprising Compounds of the Invention may be prepared using conventional diluents or excipients and techniques known in the galenic art. Thus oral dosage forms may include tablets, capsules, solutions, suspensions and the like. The term “pharmaceutically acceptable carrier” as used herein is intended to include diluents such as saline and aqueous buffer solutions. The Compounds of the Present Invention may be administered in a convenient manner such as by injection such as subcutaneous, intravenous, by oral administration, inhalation, transdermal application, intravaginal application, topical application, intranasal, sublingual or rectal administration. Depending on the route of administration, the active compound may be coated in a material to protect the compound from the degradation by enzymes, acids and other natural conditions that may inactivate the compound. In a preferred embodiment, the compound may be orally administered. In another embodiment, the compound is administered via topical application.

In certain embodiment, the Compounds of the Invention may be administered alone or in conjunction, e.g., at or about the same time or simultaneously and separately or simultaneously in an admixture, with another agent, e.g., an agent to facilitate entry or permeability of the Compounds of the Invention into the cell, e.g., an antimicrobial cationic peptide. Antimicrobial cationic peptides include peptides which contain (1) a disulfide-bonded β-sheet peptides; (2) amphipathic α-helical peptides; (3) extended peptides; or (4) loop-structured peptides. Examples of cationic peptide include but are not limited to defensins, cecropins, melittins, magainins, indolicidins, bactenecin and protegrins. Other examples of antimicrobial cationic peptides include but are not limited to human neutrophil defensin-1 (HNP-1), platelet microbicidal protein-1 (tPMP), inhibitors of DNA gyrase or protein synthesis, CP26, CP29, CP11CN, CP10A, Bac2A-NH₂ as disclosed in Friedrich et al., Antimicrob. Agents Chemother. (2000) 44(8):2086, the contents of which are hereby incorporated by reference in their entirety. Further examples of antibacterial cationic peptides include but are not limited to polymyxin e.g., polymixin B, polymyxin E or polymyxin nonapeptide. Therefore, in another embodiment, the Compounds of the Invention may be administered in conjunction with polymyxin, e.g., polymixin B, polymyxin E or polymyxin nonapeptide, preferably polymyxin B.

### Minimum Inhibitory Concentration:

### Example 1

The MIC assays are carried out in a final volume of 100 μL in 96-well clear round-bottom plates according to methods established by the Clinical Laboratory Standards Institute (CLSI). Briefly, test compound suspended in 100% DMSO (or another suitable solubilizing buffer) is added to an aliquot of media appropriate for a given pathogen to a total volume of 50 μL. This solution is serially diluted by 2-fold into successive tubes of the same media to give a range of test compound concentrations appropriate to the assay. To each dilution of test compound in media is added 50 μl of a bacterial suspension from an overnight culture growth in media appropriate to a given pathogen. Final bacterial inoculum is approximately 10⁵-10⁶ CFU/well. After growth for 18-24 hours at 37° C., the MIC is defined as the lowest concentration of antimicrobial agent that completely inhibits growth of the organism as detected by the unaided eye, relative to control for bacterial growth in the absence of added antibiotic. Ciprofloxacin is used as an antibiotic-positive control in each screening assay. Each of the bacterial cultures that are available from the American Type Culture Collection (ATCC, www.atcc.org) is identified by its ATCC number.

The experiments show that compounds of the invention, e.g., compounds set forth in formula 1.33-1.43, have a minimum inhibitory concentration (MIC) of less than 130 μg/mL against at least one of the bacteria selected from Staphylococcus aureus, Streptococcus pneumoniae, Streptococcus pyogenes, Haemophilus influenzae, Staphylococcus epidermidis, Enterococcus faecalis, and Escherichia coli.

### Binding of Ligand to Riboswitch:

### Example 2

The TPP riboswitch receptor region upstream of the tenA thiamine biosynthesis operon of B. subtilis is PCR amplified using the DNA primers 5′-TAATACGACTCACTATAGGATTCGTTTAACCACTAGGG (T7 RNA polymerase promoter and additional G residues are underlined) and 3′-TTTATGGCGAGGTGAAGG. To improve transcription efficiency with T7 RNA polymerase, these primers are designed to add two G nucleotides at the 5′-end of the natural RNA sequence. RNA constructs used for in-line probing are transcribed in vitro from PCR amplified-DNA using T7 RNA polymerase, dephosphorylated with calf intenstinal alkaline phosphatase, and 5′-³²P-end-labeled using protocols similar to those described previously in Seetharaman, S., Zivarts, M., Sudarsan, N., and Breaker, R. R., 2001 Nature Biotechnology 19, 336-341, the contents of which are hereby incorporated by reference in their entirety. For in line-probing reactions, a trace concentration of 5′-³²P-labeled RNA is incubated for ˜40 h at 25° C. in 10 μL of in-line probing buffer (50 mM Tris-HCl [pH 8.5 at 25° C.], 20 mM MgCl₂, and 100 mM KCl) containing varying concentrations of TPP or given small molecule compounds as defined for each experiment. After incubation, 10 μL of a solution containing 7 M urea and 1.5 mM EDTA is added to each in-line probing reaction and the subsequent solution is fractionated denaturating 10% polyacrlyamide gel electrophoresis (PAGE). Gels are dried and analyzed using a Storm Phospholmager (GE Healthcare). The fraction of RNA cleaved at specific sites is plotted as a function of ligand concentration change to provide an estimate of the IC₅₀ values for each compound.

The experiment shows that various Compounds of the Invention, e.g., compounds set forth in any of formulae 1.37, 1.38, 1.43 or 5.1, have a binding affinity to TPP riboswitch with an IC₅₀ value of less than, or equal to, 20 μM.

### Synthesis of the Compounds of the Invention:

General Methods.

Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. NMR data is in the delta values of major diagnostic protons, given in parts per million (ppm) relative to appropriate solvents. Conventional abbreviations for signal shape are used. Coupling constants (J), when provided, are given in Hz. For mass spectra (MS), the lowest mass major ion is reported for molecules where isotope splitting results in multiple mass spectral peaks. Solvent mixture compositions are given as volume percentages or volume ratios. In cases where the NMR spectra are complex, only diagnostic signals are reported.

### General Methods for Analytical HPLC Analysis:

### Method A

Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 98% organic (0.5 to 10.5 min); 98% organic (2 min); a gradient from 98% organic to 95% aqueous (5.5 min); 95% aqueous (1 min).

### Method B

Analytical HPLC is performed using a Zorbax C18 (15 cm×2.1 mm) column, Solvent A: acetonitrile with 0.1% formic acid, Solvent B: water with 0.1% formic acid, gradient 5% A to 85% A over 15 min.

### Method C

Analytical LCMS is performed using a YMC Combiscreen ODS-AQ, 5 μm, 4.6×50 mm column. The aqueous phase is 1% 2 mM NH₄OAc in 90:10 IPA:H₂O, 0.03% TFA in USP water. The organic phase is 1% 2 mM NH₄OAc in 90:10 IPA:H₂O, 0.03% TFA in acetonitrile. The elution profile is as follows: a gradient from 95% aqueous to 100% organic (0 to 10 min); 100% organic (2 min); a gradient from 100% organic to 95% aqueous (0.1 min); 95% aqueous (3 min).

### General Methods for Preparative HPLC:

### Method 1

Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: 100% aqueous (0 to 3 min); a gradient from 100% aqueous to 98% organic (3 to 21 min); 98% organic (1 min); a gradient from 98% organic to 95% aqueous (1 min); 95% aqueous (1 min).

### Method 2

Preparatory HPLC is performed using a Phenomenex C18 (150×30 mm) 5 μl column, 5% acetonitrile to 90% acetonitrile over 20 min, flow 20 mL/min.

### TERMS AND ABBREVIATIONS

CH₃CN=acetonitrile,
CH₂Cl₂=dichloromethane
Conc.=concentrated,
DCC=dicyclohexylcarbodiimide,
DIPEA=diisopropylethylamine
DMF=N,N-dimethylforamide,
DMA=N,N-dimethylacetamide
DMSO=dimethyl sulfoxide,
EtOAc=ethyl acetate,
Rf=retention factor
h=hour(s),
HBTU=O-Benzotriazole-N,N,N′,N′-tetramethyl-uronium-hexafluoro-phosphate,
HCl=hydrochloric acid
HPLC=high performance liquid chromatography,
K₂CO₃=potassium carbonate,
MeOH=methanol,
NaHCO₃=sodium bicarbonate,
NH₄OH=ammonium hydroxide,
POCl₃=phosphorous oxychloride,
RT=Room temperature
SOCl₂=thionyl chloride,
rt=retention time,
TFA=trifluoroacetic acid,
TLC=thin layer chromatography,
TMSBr=trimethylsilylbromide

### Example 3

### 3-((2,4-diaminopteridin-6-yl)methylamino)benzoic acid

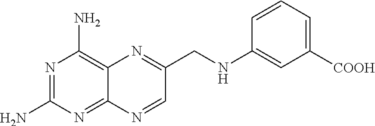

Reaction Scheme for Example 3:

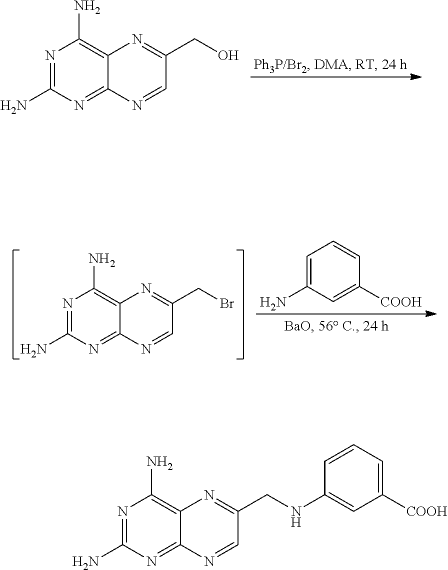

### Step 1. Preparation of (2,4-diaminopteridin-6-yl)methanol

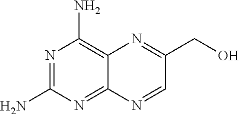

To a suspension of tetraminopyrimidine sulfate (7.14 g, 30 mmol) in water is added barium chloride (7.32 g, 30 mmol) at once. The mixture is heated at 100° C. for 10 min and cooled to RT. The solid barium sulfate is removed by filtration. The filtrate is added to a solution of 450 mL of 4 M aqueous sodium acetate solution containing dihydroxyacetone (8 g, 90 mmol) and cysteine hydrochloride monohydrate (3.63 g, 30 mmol) in a 1 liter 3-neck round bottom flask attached with a mechanical stirrer and stirred for 24 h at RT open to air. The precipitated yellow solid is filtered, washed with water, and ethanol and dried overnight in a heated vacuum oven to give 3.4 g (66%) of product. This product is further purified as per the following procedure. The yellow solid is dissolved in 10% acetic acid with aid of few drops of conc. HCl at 75° C. The hot solution is treated with activated charcoal and filtered. The filtrate is neutralized with conc. NH₄OH. The bright yellow solid is collected, washed with water, water-ethanol and finally ethanol and dried overnight in a heated vacuum oven to provide 2.8 g of the title compound (54%).

### Step 2. Preparation of 3-((2,4-diaminopteridin-6-yl)methylamino)benzoic acid

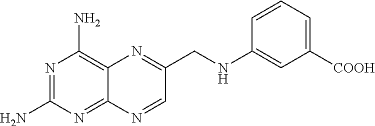

To a solution of triphenylphosphene (408 mg, 1.03 mmol) in anhydrous DMA (1 mL) is added bromine dropwise at 0° C. (0.08 mL, 1.03 mmol) under N₂ atmosphere. After stirring for 5 additional minutes, (2,4-diaminopteridin-6-yl)methanol (100 mg, 0.33 mmol) is added at once and the reaction mixture is stirred at RT for 18 h. Barium oxide (100 mg, 0.65 mmol) is then added to the reaction mixture followed by 3-aminobenzoic acid (107 mg, 0.78 mmol) at RT. The reaction mixture is then heated at 56° C. and stirred at that temperature for 24 h and cooled to RT. The mixture is diluted with methylene chloride (5 mL) and the resulting brownish precipitate is filtered. The solids are washed with water and methanol. The solids are then taken in methanol and heated at reflux for 2 h. After cooling to RT, the solids are filtered again and dried overnight in a heated vacuum oven to give 45 mg product (Yield: 26.9%) as a brownish yellow solid. ¹H NMR (500 MHz, DMSO-d₆) δ 4.6 (br s, 2H), 6.7 (s, 1H), 6.9-7.0 (d, 1H), 7.15-7.3 (m, 2H), 7.38 (br s, 2H), 8.6-8.7 (br s, 1H), 8.9 (s, 1H), 9.3-9.4 (m, 2H), 12.8 (br s, 1H); LC-MS m/z 312 (MH+), retention time 11.48 min., HPLC Method B.

The compounds of Examples 4-9 are prepared using the procedure as described for Example 3.

### Example 4

### 2-((2,4-diaminopteridin-6-yl)methylamino)benzoic acid

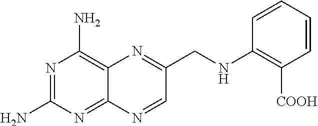

The title compound is prepared using a procedure similar to that of Example 3 except 2-aminobenzoic acid is used in place of 3-aminobenzoic acid. ¹H NMR (500 MHz, DMSO-d₆) δ 4.6 (s, 2H), 6.55-6.60 (m, 1H), 6.7-6.8 (m, 31-1), 7.4-7.5 (m, 2H), 7.8 (d, 1H), 8.0 (br s, 1H), 8.7 (s, 1H), 8.9 (br s, 1H), 12.9 (br s, 1H), LC-MS m/z 312 (MH+), retention time 13.17 min., HPLC Method B.

### Example 5

### 4-((2,4-diaminopteridin-6-yl)methylamino)-2-hydroxybenzoic acid

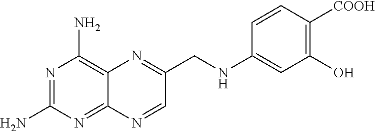

The title compound is prepared using a procedure similar to that of Example 3 except 4-amino-2-hydroxybenzoic acid is used in place of 3-aminobenzoic acid. ¹H NMR (500 MHz, DMSO-d₆) δ 4.6 (s, 2H), 6.1 (d, 1H), 6.3 (m, 1H), 7.2 (s, 1H), 7.5 (br s, 2H), 8.6 (br s, 1H), 8.9 (s, 1H), 9.4 (m, 2H), 11.5 (br s, 1H), 12.9-13.1 (br s, 1H). LC-MS m/z 328 (MH+), retention time 11.42 min., HPLC Method B.

### Example 6

### 2-(4-((2,4-diaminopteridin-6-yl)methylamino)-phenyl)acetic acid

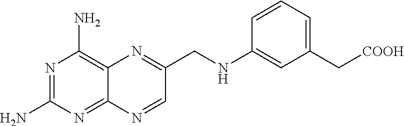

The title compound is prepared using a procedure similar to that of Example 3 except (4-aminophenyl)acetic acid is used in place of 3-aminobenzoic acid. ¹H NMR (500 MHz, MeOH-d₄) δ, 4.6 (s, 2H), 5.1 (s, 1H), 6.6-6.7 (m, 2H), 6.8 (m, 1H), 7.1-7.2 (m, 1H), 8.9 (s, 1H). LC-MS m/z 326 (MH+).

### Example 7

### 6-((4-(trifluoromethylsulfonyl)phenylamino)methyl)pteridine-2,4-diamine

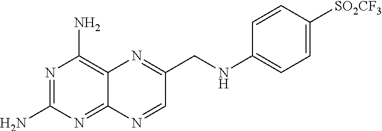

The title compound is prepared using a procedure similar to that of Example 3 and by using 4-(trifluoromethylsulfonyl)aniline, which is prepared following the procedure reported in Bioorganic and Medicinal Chemistry Letters, 1995, 5(20), 2303-8, the contents of which are incorporated herein by reference in their entirety. LC-MS m/z 400 (MH+), retention time 15.45 min., HPLC Method B.

### Example 8

### 3-((2,4-diaminopteridin-6-yl)methylamino)benzylphosphonic acid

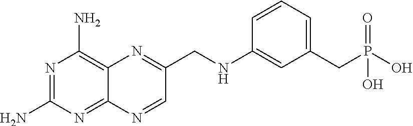

The title compound is prepared using a procedure similar to that of Example 3 and by using diethyl 3-aminobenzylphosphonate, which is prepared as described below. LC-MS m/z 362 (MH+), retention time 2.21 min., HPLC Method B.

### Step 1. Preparation of diethyl 3-nitrobenzylphosphonate

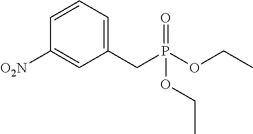

A mixture of 3-nitrobenzyl bromide (2.16 g, 1 mmol) and triethylphosphite (1.66 g, 1 mmol) in DMF (10 mL) is heated at 90° C. under for 16 h under nitrogen atmosphere. The reaction mixture is cooled to RT and diluted with ethyl acetate. The organic layer is washed with water (2×) and brine. After drying (Na₂SO₄), the solvent is concentrated.

The crude product (2.56 g) (TLC, 80% ethyl acetate/hexanes, Rf 0.35) is used as it is for the next step.

### Step 2. Preparation of diethyl 3-aminobenzylphosphonate

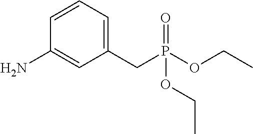

The product from step 1 (2.56 g, 9.4 mmol) is dissolved in 10 mL of ethyl alcohol and added to a round bottom flask containing 250 mg of 10% palladium/carbon catalyst. The mixture is purged with H₂ and stirred at RT for 16 h under a H₂ atmosphere using a balloon. Upon completion of reaction by TLC analysis, the mixture is then passed through a pad of celite and washed with ethanol. The filtrate is concentrated. The pure product (1.45 g, 64%) is isolated by silica gel column chromatography using 50 to 75% CH₃CN in CH₂Cl₂. ¹H NMR (500 MHz, DMSO-d₆) δ 6.9-7.0 (t, 1H), 6.7 (s, 1H), 6.4-6.5 (m, 2H), 5.3-5.6 (br, 2H), 3.9 (m, 4H), 3.4 (s, 2H), 1.2 (t, 6H).

### Example 9

### 4-((2,4-diaminopteridin-6-yl)methylamino)benzenesulfonic acid

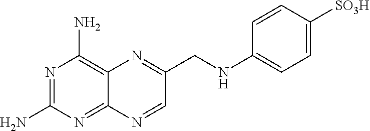

The title compound is prepared using a procedure similar to that of Example 3 except 4-aminobenzenesulfonic acid is used in place of 3-aminobenzoic acid. LC-MS m/z 348 (MH+), retention time 14.71 min., HPLC Method B.

### Examples 10 and 11

### Example 10

### 2-amino-2-(4-((2,4-diaminopteridin-6-yl)methylamino)phenyl)acetic acid

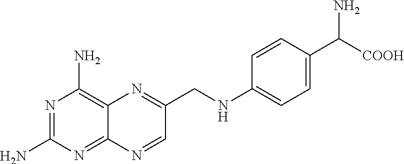

¹H NMR NMR (500 MHz, D₂O) δ: 4.7 (s, 1H), 5.0 (s, 2H), 6.88-6.9 (d, 1H), 7.15-7.20 (d, 1H), 8.7 (s, 1H). LC-MS m/z 341 (MH+), retention time 2.51 min., HPLC Method B.

### Example 11

### 2-amino-2-(4-((2,4-diaminopteridin-7-yl)methylamino)phenyl)acetic acid

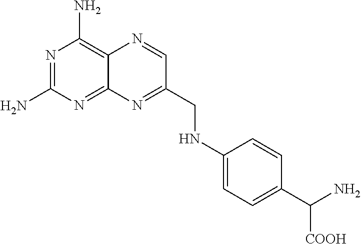

LC-MS m/z 341 (MH+), retention time 2.51 min., HPLC Method B.

The title compounds are prepared using a procedure similar to that of Example 3 and by using rac-2-(4-aminophenyl)-2-(Cert-butoxycarbonylamino)acetic acid. In the final workup, the tert-butoxycarbonyl group is removed using trifluoroacetic acid in methylene chloride (50/50 by volume). The crude reaction mixture from the deprotection is purified by preparative HPLC (Method 2). The major component (rt=3.79 min.) is 2-amino-2-(4-((2,4-diaminopteridin-6-yl)methylamino)phenyl)acetic acid and the minor component is 2-amino-2-(4-((2,4-diaminopteridin-7-yl)methylamino)phenyl)acetic acid (rt=4.69 min.).

### Example 12

### Procedure B

### 4-(N-((2,4-diaminopteridin-6-yl)methyl)-N-methylamino)benzoic acid

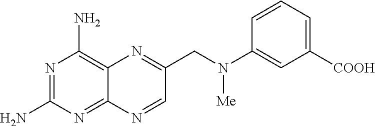

To a suspension of commercially available 6-(bromomethyl)pteridine-2,4-diamine hydrochloride (50 mg, 0.154 mmol) in DMA (2 mL) is added 3-(N-methylamino)benzoic acid (3 equivalents) and potassium carbonate (41 mg, 0.29 mmol, 2 equiv) at once. The mixture is heated at 70° C. for overnight and cooled to RT. The reaction mixture is diluted with dichloromethane. The precipitated solid is filtered, washed with water and hot methanol to give the desired product as brown solid (31%). ¹H NMR (500 MHz, MeOH-d₄ with a drop of TFA-d) δ 3.2 (s, 3H), 5.4 (s, 2H), 6.7 (d, 1H), 6.9 (d, 1H), 7.1-7.3 (m, 2H), 8.6 (s, 1H); LC-MS m/z 326 (MH+), retention time 6.88 min., HPLC Method B.

The compounds of Examples 13-20 are prepared using procedure as described for Example 12.

### Example 13

### Diethyl 4-((2,4-diaminopteridin-6-yl)methylamino)phenylphosphonate

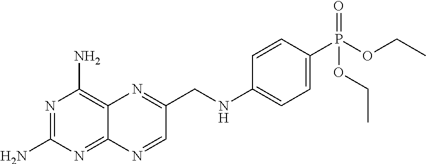

The title compound is prepared using a procedure similar to that of Example 12 except diethyl 4-aminobenzylphosphonate is used in place of 3-(N-methylamino)benzoic acid. ¹NMR (500 MHz, MeOH-d₄) δ 1.7 (m, 6H), 4.0-4.2 (m, 4H), 4.6 (s, 2H), 6.8 (m, 2H), 7.5 (m, 2H), 8.75 (s, 1H).

### Example 14

### 6-((3-aminophenylamino)methyl)pteridine-2,4-diamine

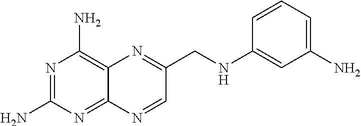

The title compound is prepared using a procedure similar to that of Example 12 except 1,3-benzenediamine is used in place of 3-(N-methylamino)benzoic acid. LC-MS m/z 282 (MH+), retention time 3.89 min., HPLC Method B.

### Example 15

### 3-((2,4-diaminopteridin-6-yl)methylamino)-N-(methylsulfonyl)benzamidediamine

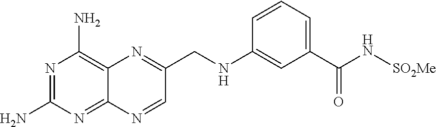

The title compound is prepared using a procedure similar to that of Example 12 and by using 3-amino-N-(methylsulfonyl)benzamide which is prepared following the procedure reported in Bioorganic and Medicinal Chemistry Letters, 1995, 5(20), 2303-8, the contents of which are incorporated herein by reference in their entirety. LC-MS m/z 389.5 (MH+), retention time 3.15 min., HPLC Method B.

### Example 16

### 3-(4-((2,4-diaminopteridin-6-yl)methylamino)phenyl)-2-hydroxyacrylic acid

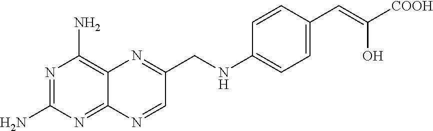

The title compound is prepared using a procedure similar to that of Example 12 and by using 3-(4-aminophenyl)-2-oxopropanoic acid which is prepared following the procedure reported in Synthesis, 1992, 793-797, the contents of which are incorporated herein by reference in their entirety. LC-MS m/z 354 (MH+), retention time 2.31 min., HPLC Method B.

### Example 17

### methyl-4-((2,4-diaminopteridin-6-yl)methylamino)-2-((methoxycarbonyl)methoxy)-benzoate

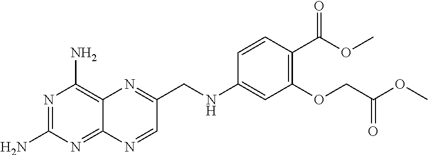

The title compound is prepared using a procedure similar to that of Example 12 except methyl 4-amino-2-(2-methoxy-2-oxoethoxy)benzoate is used in place of 3-(N-methylamino)benzoic acid. LC-MS m/z 414 (MH+), retention time 8.07 min., HPLC Method B.

### Example 18

### 4-((2,4-diaminopteridin-6-yl)methylamino)-2-(carboxymethoxy)benzoic acid

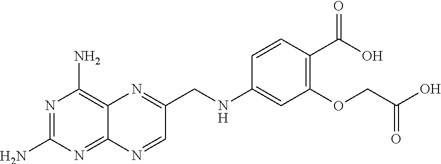

The title compound is prepared using a procedure similar to that of Example 12 except 4-amino-2-(carboxymethoxy)benzoic acid is used in place of 3-(N-methylamino)benzoic acid. NMR (500 MHz, MeOH-d₄) δ 4.1 (s, 2H), 6.2-6.3 (m, 1H), 6.9 (s, 1H), 7.5 (d, 1H), 8.5 (s, 1H).

### Example 19

### 4-((2,4-diaminopteridin-6-yl)methylamino)phenylphosphonic acid

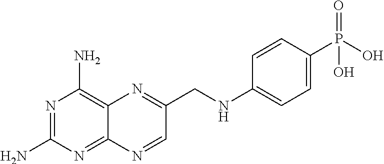

The title compound is prepared using a procedure similar to that of Example 12 and by using 4-aminophenylphosphonic acid which is prepared following the procedure reported in Journal of Medicinal Chemistry, 2001, 44, 340-349, the contents of which are incorporated herein by reference in their entirety. LC-MS m/z 348 (MH+), retention time 2.48 min., HPLC Method B.

### Example 20

### 3-((2,4-diaminopteridin-6-yl)methylamino)phenylphosphonic acid

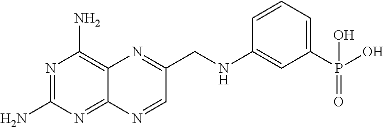

The title compound is prepared using a procedure similar to that of Example 12 and by using 3-aminophenylphosphonic acid. ¹H NMR (500 MHz, DMSO-d₆₊ drops of TFA) 4.55 (s, 2H), 6.7-6.75 (m, 1H), 6.8-6.85 (m, 1H), 7.1-7.2 (m, 2H), 8.7 (s, 1H).

### Example 21

### 4-((2,4-Diaminopteridin-6-yl)methylamino)benzylphosphonic acid

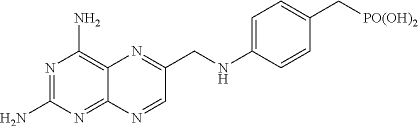

Reaction Scheme for Example 21

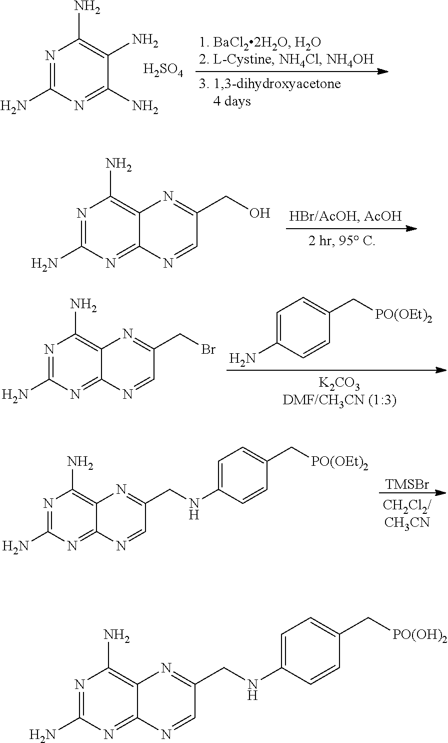

### Step 1 Preparation of 6-(bromomethyl)pteridine-2,4-diamine

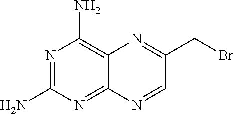

6-(Bromomethyl)pteridine-2,4-diamine is prepared starting from (2,4-diaminopteridin-6-yl)methanol [Step 1, Example 3] following the procedure described in J. Med. Chem., 1968, 11, 1238-1241. Crude 6-(bromomethyl)pteridine-2,4-diamine is used in the next step.

### Step 2 Preparation of diethyl 4-((2,4-diaminopteridin-6-yl)methylamino)benzyl phosphonate

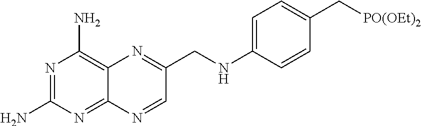

Crude 6-(bromomethyl)-2,4-pteridinediamine (60 mg, 0.235 mmol), diethyl 4-aminobenzylphosphonate (0.302 g, 1.24 mmol), and K₂CO₃ (428 mg, 3.1 mmol) are dissolved in a DMF (1 mL), CH₃CN (3 mL) mixture and heated to 70° C. overnight (15 h). The crude reaction mixture is filtered and washed with MeOH (30 mL). The filtrate is concentrated by rotoevaporation, dissolved in a minimum amount of MeOH (5 mL), and filtered again by syringe filter (0.45 um) for purification by prep HPLC using Method 1. The desired fractions are combined and concentrated to give 10 mg of diethyl 4-((2,4-diaminopteridin-6-yl)methylamino)benzyl phosphonate (Yield; 10%). The product is used in the next step with out any further purification. LC-MS m/z 418.1 [M+H]⁺, retention time 2.29 min., HPLC Method C.

### Step 3 Preparation of 4-((2,4-Diaminopteridin-6-yl)methylamino)benzylphosphonic acid

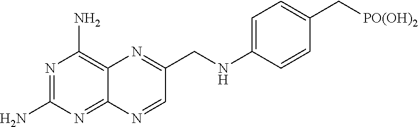

Diethyl 4-((2,4-diaminopteridin-6-yl)methylamino)benzyl phosphonate (10 mg, 0.024 mmol) from Step 3 is dissolved in DCM (1.5 mL), CH₃CN (0.5 mL) and TMSBr (0.5 mL, 3.8 mmol) is added drop wise to this mixture at RT, and stirred for 25 h. LCMS showed mono phosphonate is present in the phosphonic acid crude reaction mixture and additional TMSBr (0.5 mL, 3.8 mmol) is added and stirred for 28 h. A trace amount of mono ester is still present, however, the reaction mixture is concentrated and the residue is dissolved in MeOH (1 mL) and conc. HCl (0.2 mL) is added and the mixture is stirred for 30 min. The solvent is evaporated and the crude acid is dissolved in a minimum amount of hot MeOH (0.5 mL). EtOAc is added dropwise until the product precipitated. The product is filtered and washed with EtOAc (2 mL) to provide 6.7 mg of {4-[(2,4-Diamino-pteridin-6-ylmethyl)-amino]-benzyl}-phosphonic acid (Yield: 77%). ¹H NMR (400 MHz, MeOH-d₄) δ 3.37 (s, 2H), 5.01 (s, 2H), 7.51 (m, 4H), 8.92 (s, 1H); LC-MS m/z 362.2 [MH+], retention time 0.92 min., HPLC Method C.

### Example 22

### 2-(4-((2,4-diaminopteridin-6-yl)methylamino)phenyl)acetic acid

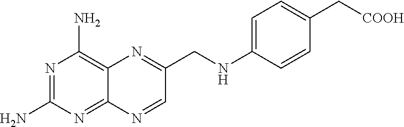

Reaction Scheme for Example 22

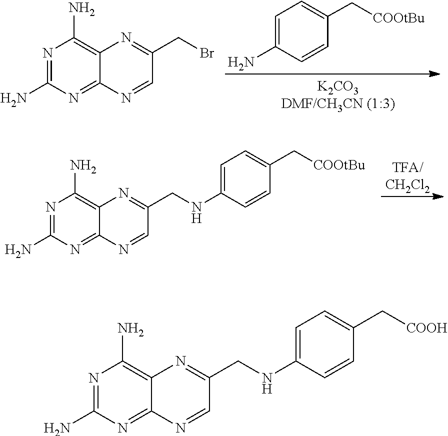

### Step 1 Preparation of tert-butyl 2-(4-((2,4-diaminopteridin-6-yl)methylamino) phenyl)acetate

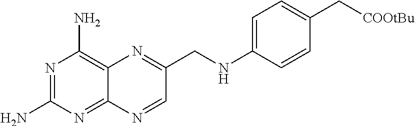

Crude 6-(bromomethyl)-2,4-pteridinediamine (Step 1, Example 19)(80 mg, 0.392 mmol), tert-butyl 2-(4-aminophenyl)acetate (228 mg, 1.12 mmol), and K₂CO₃ (542 mg, 3.9 mmol) are dissolved in a DMF (1 mL), CH₃CN (3 mL) mixture and heated to 70° C. overnight (15 h). The crude reaction mixture is filtered and washed with MeOH (30 mL). The filtrate is concentrated by rotoevaporation, dissolved in minimum amount of MeOH (5 mL), and filtered again by syringe filter (0.45 um) for purification by prep HPLC using Method 1. The desired fractions are combined and concentrated to give 15 mg of tert-butyl 2-(4-((2,4-diaminopteridin-6-yl)methylamino) phenyl)acetate (Yield; 10%).

### Step 2 Preparation of 2-(4-((2,4-diaminopteridin-6-yl)methylamino)phenyl)acetic acid

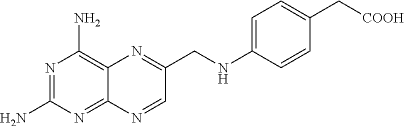

Tert-butyl 2-(4-((2,4-diaminopteridin-6-yl)methylamino)phenyl)acetate from Step 1 (15 mg, 0.039 mmol) is dissolved in DCM (1 mL) and trifluoroacetic acid (1.0 mL) and stirred the reaction mixture is stirred at RT for 1.5 h. The reaction mixture is concentrated to provide 11.1 mg of 2-(4-((2,4-diaminopteridin-6-yl)methylamino)phenyl)acetic acid (Yield: 87%). ¹H NMR (400 MHz, MeOH-d₄) δ 3.69 (s, 2H), 4.91 (s, 2H), 7.35 (m, 4H), 8.67 (s, 1H); LC-MS m/z 323.9 (M−H), retention time 1.38 min., HPLC Method C.

### Example 23

### Methyl 2-(4-(6-((4-amino-2-methylpyrimidin-5-yl)methylamino)pyrimidin-4-yloxy)phenyl)acetate

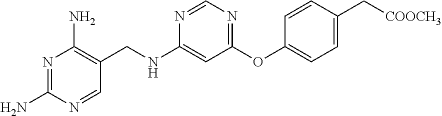

Reaction Scheme for Example 21

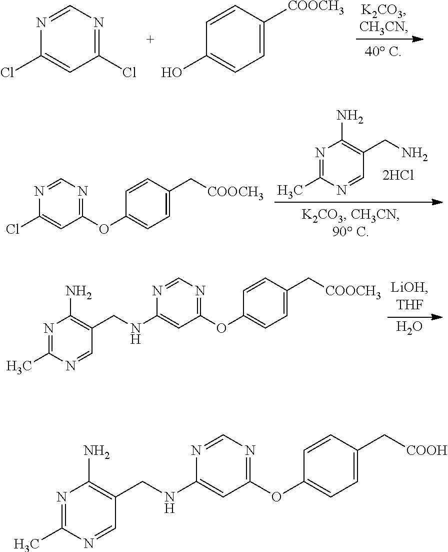

### Step 1 Preparation of methyl 2-(4-(6-chloropyrimidin-4-yloxy)phenyl)acetate

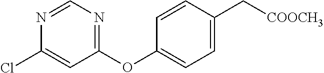

A suspension of 4,6-dichloropyrimidine (149 mg, 1.0 mmol), methyl 4-hydroxybenzoate (166 mg, 1.0 mmol), and potassium carbonate (690 mg, 5.0 mmol) in acetonitrile (6 mL) is stirred at 60° C. for 24 h. The reaction mixture is used in the next step without any workup or purification. LC-MS m/z 278.9 [M+H]⁺, retention time 4.06 min., HPLC Method C.

### Step 2 Preparation of methyl 2-(4-(6-((4-amino-2-methylpyrimidin-5-yl)methylamino) pyrimidin-4-yloxy)phenyl)acetate

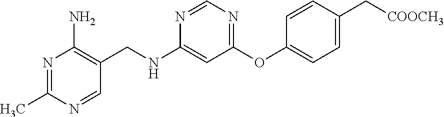

To the crude reaction mixture from Step 1, is added 5-(aminomethyl)-2-methylpyrimidin-4-amine dihydrochloride (211 mg, 1.0 mmol) in acetonitrile (2 mL). The reaction mixture is heated to reflux at 95° C. for 24 h. The reaction mixture is cooled to RT, and the volatiles are removed under reduced pressure. The resulting residue is purified by preparative HPLC (Method 1). Methyl 2-(4-(6-((4-amino-2-methylpyrimidin-5-yl)methylamino)pyrimidin-4-loxy)phenyl)acetate (37 mg) is isolated (Yield: 9.7% for 2 steps). ¹H NMR (400 MHz, D₂O-d₆) δ: 2.50 (s, 3H), 3.68 (s, 3H), 3.77 (s, 2H), 4.46 (s, 2H), 5.87 (s, 1H), 7.19 (d, 2H), 7.39 (d, 2H), 7.93 (s, 1H), 8.46 (s, 1H); LC-MS m/z 381.3 [M+H]⁺, retention time 4.02 min., HPLC Method C.

### Example 24

### 2-(4-(6-((4-amino-2-methylpyrimidin-5-yl)methylamino)pyrimidin-4-yloxy)phenyl)acetic acid

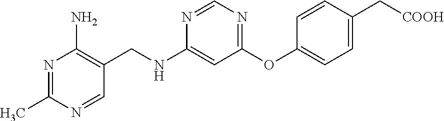

To a stirred solution of methyl 2-(4-(6-((4-amino-2-methylpyrimidin-5-yl)methylamino) pyrimidin-4-yloxy)phenyl)acetate (31 mg, 0.08 mmol) in THF (2 mL)/water (1 mL), is added lithium hydroxide (33 mg, 0.8 mmol). The reaction mixture is stirred overnight at 70° C. The reaction mixture is neutralized with dilute HCl and concentrated to remove volatiles. The resulting residue is purified by preparative HPLC (Method 1). 2-(4-(6-((4-amino-2-methylpyrimidin-5-yl)methylamino)pyrimidin-4-yloxy)phenyl)acetic acid is isolated on evaporation (19.4 mg, yield: 65.0%). ¹H NMR (400 MHz, D₂O-d₆) δ: 2.52 (s, 3H), 3.77 (s, 2H), 4.70 (s, 2H), 5.86 (s, 1H), 7.17 (d, 2H), 7.39 (d, 2H), 7.91 (s, 1H), 8.33 (s, 1H); LC-MS m/z 367.3 [M+H], retention time 1.60 min., HPLC Method C.

### Example 25

### Diethyl 2-(4-((1-amino-2-methylpyrimidin-5-yl)methylamino)-4-oxobutanamido) ethylphosphonate

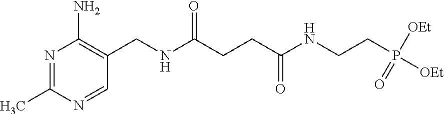

Reaction Scheme for Example 25

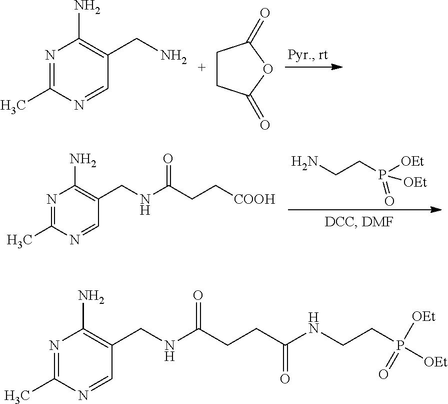

### Step 1 Preparation of 4-((4-amino-2-methylpyrimidin-5-yl)methylamino)-4-oxobutanoic acid

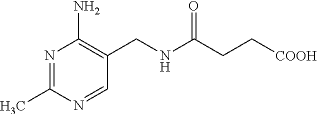

A suspension of 5-(aminomethyl)-2-methylpyrimidin-4-amine (690 mg, 5.0 mmol) and succinic anhydride (500 mg, 5.0 mmol) in pyridine (15 ml) is stirred at RT overnight. The reaction mixture is concentrated to remove pyridine. 4-((4-amino-2-methylpyrimidin-5-yl)methylamino)-4-oxobutanoic acid is obtained as a white solid after removal of pyridine using a high vacuum pump (1.1 g, yield: 100%).

### Step 2 Preparation of diethyl 2-(4-((4-amino-2-methylpyrimidin-5-yl)methylamino-4-oxobutanamido)ethylphosphonate

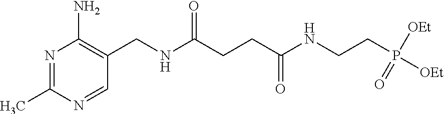

To a stirred suspension of 4-((4-amino-2-methylpyrimidin-5-yl)methylamino)-4-oxobutanoic acid from step 1 (100 mg, 0.42 mmol) in DMF (5 mL), is added DCC (94 mg, 0.46 mmol). The reaction mixture is stirred at RT for 20 min. Then diethyl 2-aminoethylphosphonate (69 mg, 0.38 mmol) is added and reaction mixture is stirred at RT for 24 h. The reaction is quenched by addition of water (1 mL) and reaction mixture is concentrated. The crude residue is purified by preparative TLC using MeOH:DCM:NH₄OH (13:85:2) as eluent to obtain 21 mg of the desired product (Yield: 12.5%). ¹H NMR (400 MHz, MeOH-d₄) δ: 1.35 (m, 6H), 2.06 (m, 2H), 2.52 (s, 3H), 2.54 (s, 4H), 3.43 (m, 2H), 4.12 (m, 4H), 4.24 (s, 2H), 8.06 (s, 1H); LC-MS m/z 402.3 [M+H], retention time 4.04 min., HPLC Method A.

### Example 26

### (((2-(4-(((4-amino-2-methylpyrimidin-5-yl)methyl)amino)-4-oxobutanamido)ethyl)phosphoryl)bis(oxy))bis(methylene)bis(2,2-dimethylpropanoate)

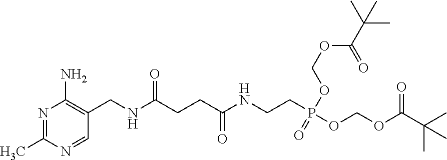

Reaction Scheme for Example 26

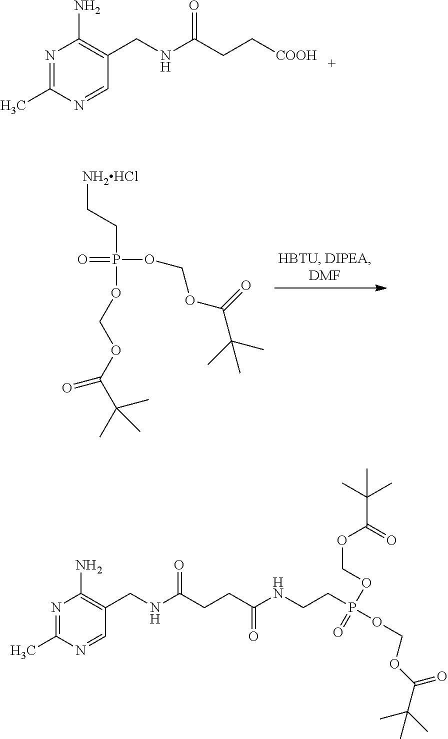

To a stirred solution of 4-((4-amino-2-methylpyrimidin-5-yl)methylamino)-4-oxobutanoic acid (90 mg, 0.38 mmol) in DMF (5 mL), is added DIPEA (97 mg, 0.75 mmol) followed by HBTU (161 mg, 0.43 mmol). After stirring the reaction mixture at RT for 30 min, ((2-aminoethyl)phosphoryl)bis(oxy)bis(methylene)bis(2,2-dimethylpropanoate)hydrochloride (107 mg, 0.30 mmol) is added and the reaction mixture is stirred at RT for 24 h. The reaction is quenched with water (1 mL) and reaction mixture is concentrated. The crude residue is resuspended in water (10 mL) and extracted with EtOAc (3×20 mL). The organic phase is combined, dried over Na₂SO₄ and filtered. The filtrate is concentrated to provide a crude oil which is purified by preparative TLC using EtOAc:DCM (3:7) as eluent to obtain 13.3 mg of the desired product (Yield: 6.1%). ¹H NMR (400 MHz, MeOH-d₄) δ: 1.25 (s, 18H), 2.17 (m, 2H), 2.40 (s, 3H), 2.51 (s, 4H), 3.42 (m, 2H), 4.22 (s, 2H), 5.70 (m, 4H), 7.94 (s, 1′-1); LC-MS m/z 574.3 [M+H], retention time 6.34 min., HPLC Method A.

### Example 27

### Methyl 4-(((2,4-diaminopteridin-6-yl)methyl)(methyl)amino)-2-(2-methoxy-2-oxoethoxy)benzoate

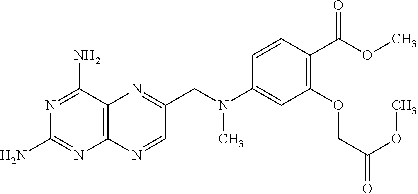

Reaction Scheme for Example 27:

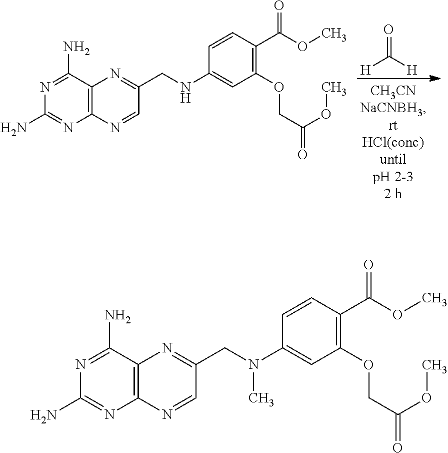

To a suspended solution of methyl 4-(((2,4-diaminopteridin-6-yl)methyl)amino)-2-(2-methoxy-2-oxoethoxy)benzoate (prepared in Example 17, 116 mg, 0.28 mmol), formaldehyde (9.66 mg, 46.8 mmol), and sodium cyanoborohydride (72 mg, 1.1 mmol) in CH₃CN (50 mL) is added concentrated HCl at room temperature until the solution is pH=2. After 2 h, the reaction mixture is concentrated and the residual material is dissolved in DMSO and purified by preparative HPLC (Method 2). Lyophilization of combined pure fractions affords desired product (15 mg, 12%) as a yellow solid. ¹H NMR (400 MHz, DMSO-d₆) δ 3.24 (s, 3H), 3.64 (s, 3H), 3.69 (s, 3H), 4.82 (s, 2H), 4.86 (s, 2H), 6.20 (s, 1H), 6.48 (d, 1H), 7.50 (br s, 1H), 7.62 (d, 1H), 8.50 (br s, 1H), 8.71 (s, 1H), 8.91 (br s, 1H), 9.19 (br s, 1H); LC-MS m/z 428.0 [M+H]⁺, retention time 3.88 min.

### Example 28

### 3-(((2,4-Diaminopteridin-6-yl)methyl)(methyl)amino)benzamide

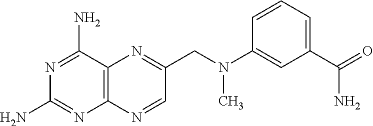

Reaction Scheme for Example 28:

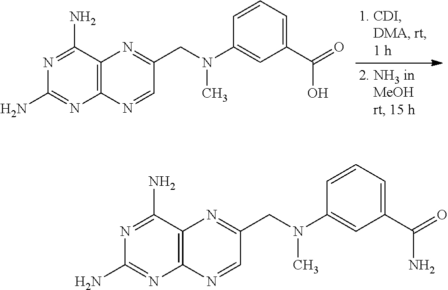

A mixture of 3-(((2,4-diaminopteridin-6-yl)methyl)(methyl)amino)benzoic acid (prepared in Example 12, 30 mg, 0.092 mmol), and N,N′-carbonyldiimidazole (22 mg, 0.135 mmol) in DMA (2 mL) is stirred at room temperature for 1 h. Ammonia (2 N in methanol, 1 mL) is added and the resulting solution is stirred at room temperature for 15 h. EtOAc is slowly added to induce precipitation. The product is collected by filtration and lyophilized to obtain the desired product (8 mg, 27%) as a yellow solid. ¹H NMR (400 MHz, DMSO-d₆) δ 3.11 (s, 3H), 4.73 (s, 2H), 6.60 (br s, 2H), 6.92 (d, 1H), 7.14 (d, 1H), 7.21 (dd, 1H), 7.26 (s, 1H), 7.39 (s, 1H), 7.44 (br s, 1H), 7.66 (s, 1H), 7.89 (s, 1H), 8.57 (s, 1H). LC-MS m/z 325.0 [M+H]⁺, retention time 3.26 min.

### Example 29

### 3-(3-(((2,4-Diaminoquinazolin-7-yl)methyl)(methyl)amino)phenyl)propanoic acid

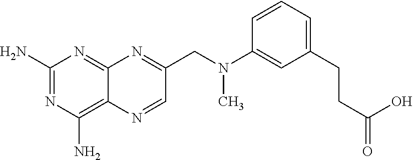

Reaction Scheme for Example 29:

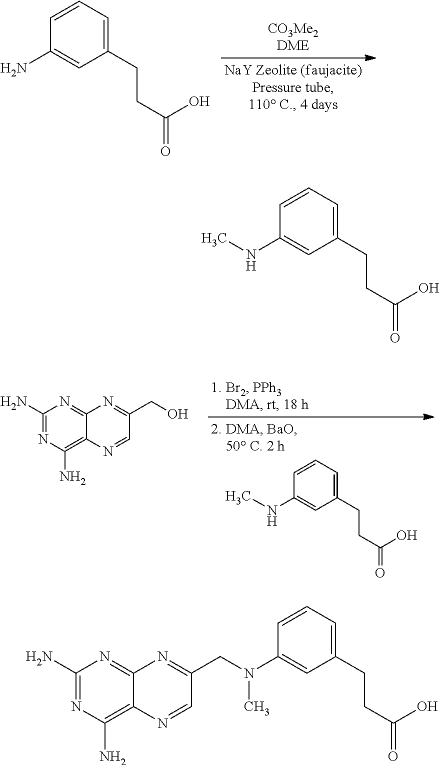

### Step 1 Preparation of 3-(3-(methylamino)phenyl)propanoic acid

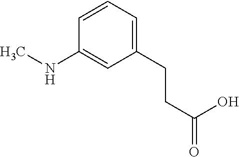

A mixture of 3-(3-aminophenyl)propanoic acid (commercial, 206 mg, 1.2 mmol), NaY Zeolite (400 mg), dimethoxyethane (2 mL) and dimethyl carbonate (8 mL) is heated in a pressure tube at 110° C. for 4 days. The reaction mixture is filtered through a celite pad and the filtrate is concentrated under reduced pressure to dryness. The product is used the next step without further purification.

### Step 2 Preparation of 3-(3-(((2,4-diaminoquinazolin-7-yl)methyl)(methyl)amino)phenyl)propanoic acid

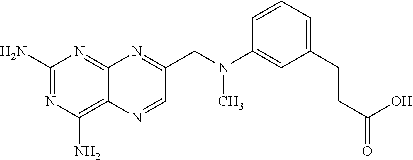

The title compound is prepared using a procedure similar to that of Example 3 except 3-(3-(methylamino)phenyl)propanoic acid is used in place of 3-aminobenzoic acid. The residual material is dissolved in DMSO and purified by preparative HPLC (Method 2). Lyophilization of combined pure fractions affords the desired product as a red gummy solid. ¹H NMR (400 MHz, DMSO-d₆) δ 2.47 (t, 2H), 2.73 (t, 2H), 3.13 (s, 3H), 4.77 (s, 2H), 6.53 (d, 1H), 6.62 (d, 1H), 6.68 (s, 1H), 7.06 (dd, 1H), 7.54 (br s, 1H), 8.63 (br s, 1H), 8.68 (s, 1H), 9.14 (s, 1H), 9.28 (s, 1H). LC-MS m/z 354.1 [M+H]⁺, retention time 3.59 min.

### Example 30

### 2-(3-(((2,4-Diaminoquinazolin-7-yl)methyl)(methyl)amino)phenoxy)acetic acid

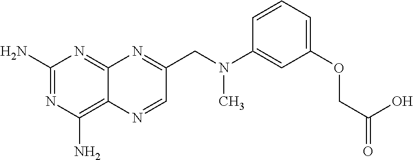

Reaction Scheme for Example 30:

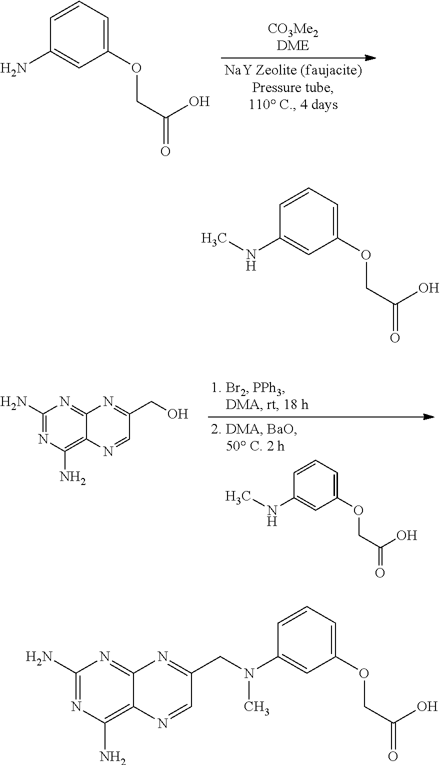

The title compound is prepared using a procedure similar to that of Example 29 except 2-(3-aminophenoxy)acetic acid (purchased from Cambridge) is used in place of 3-(3-aminophenyl)propanoic acid. ¹H NMR (400 MHz, DMSO-d₆) 3.14 (s, 3H), 4.58 (s, 2H), 4.77 (s, 2H), 6.20 (d, 1H), 6.27 (s, 1H), 6.41 (d, 1H), 7.06 (dd, 1H), 7.62 (br s, 1H), 8.58 (br s, 1H), 8.66 (s, 1H), 9.09 (s, 1H), 9.26 (s, 1H). LC-MS m/z 356.0 [M+H]⁺, retention time 3.45 min.

### Example 31

### 2-(3-(((2,4-Diaminopteridin-6-yl)methyl)(methyl)amino)phenoxy)acetamide

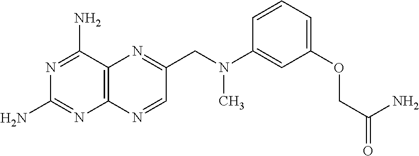

Reaction Scheme for Example 31:

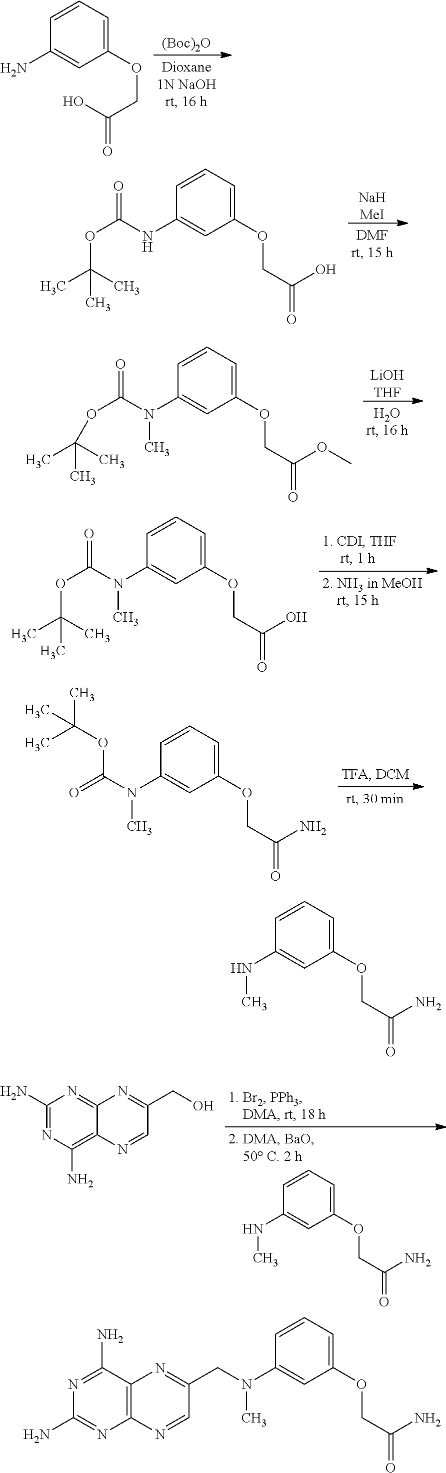

### Step 1 Preparation of 2-(3-((tert-butoxycarbonyl)amino)phenoxy)acetic acid

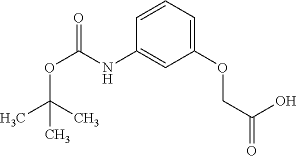

A mixture of 2-(3-aminophenoxy)acetic acid (145 mg, 0.86 mmol), di-tert-butyl dicarbonate (302 mg, 1.38 mmol), NaOH (1N, 1 mL) in dioxane is stirred at room temperature for 16 h. The reaction mixture is concentrated and the residual material is made acidic with 1N HCl (3 mL) and extracted with EtOAc. The organic layer is dried over Na₂SO₄, filtered, and concentrated under reduced pressure to obtain product (212 mg, 91%) as an off white solid. This compound is used as is without further purification.

### Step 2 Preparation of methyl 2-(3-((tert-butoxycarbonyl)(methyl)amino)phenoxy)acetate

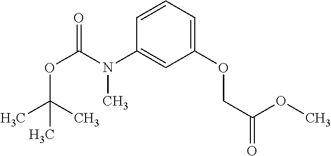

To a solution of 2-(3-((tert-butoxycarbonyl)amino)phenoxy)acetic acid (212 mg, 0.79 mmol) in DMF (4 mL) is added NaH (70 mg, 1.74 mmol) at room temperature and stirred for 20 min. To this mixture, methyl iodide (338 mg, 2.38 mmol) is added and the mixture is stirred for another 15 h. The reaction is quenched with H₂O (10 mL) and extracted with EtOAc. The organic layer is dried over Na₂SO₄, filtered, and concentrated under reduced pressure to obtain product (221 mg, 94%). This compound is used without further purification.

### Step 3 Preparation of 2-(3-((tert-butoxycarbonyl)(methyl)amino)phenoxy)acetic acid

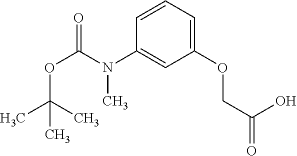

A mixture of methyl 2-(3-((tert-butoxycarbonyl)(methyl)amino)phenoxy)acetate (221 mg, 0.75 mmol) and LiOH (90 mg, 3.75 mmol) in THF (5 mL) and H₂O (5 mL) is stirred at room temperature for 16 h. The reaction mixture is concentrated and the residual material is made acidic with 1N HCl (3 mL) and extracted with EtOAc. The organic layer is dried over Na₂SO₄, filtered, and concentrated under reduced pressure to obtain product (210 mg, quantitative). This compound is used without further purification.

### Step 4 Preparation of tert-butyl (3-(2-amino-2-oxoethoxy)phenyl)(methyl)carbamate

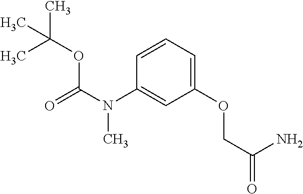

A mixture of 2-(3-((tert-butoxycarbonyl)(methyl)amino)phenoxy)acetic acid (210 mg, 0.75 mmol), and N,N′-carbonyldiimidazole (240 mg, 1.48 mmol) in THF (15 mL) is stirred at room temperature for 1 h. Ammonia (7 N in methanol, 2 mL) is added and the resulting solution is stirred at room temperature for 10 min. The reaction mixture is concentrated and H₂O is added and the mixture is extracted with EtOAc. The organic layer is dried over Na₂SO₄, filtered, and concentrated under reduced pressure. The residue is purified by BIOTAGE flash column chromatography using a gradient from 0 to 100% EtOAc in hexane as eluent. Desired product is isolated (145 mg). LC-MS m/z 280.7 [M+H]⁺, retention time 4.82 min.

### Step 5 Preparation of 2-(3-(methylamino)phenoxy)acetamide

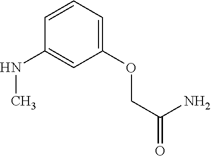

Tert-butyl (3-(2-amino-2-oxoethoxy)phenyl)(methyl)carbamate (145 mg, 0.51 mmol) is stirred in TFA (2 mL) and DCM (2 mL) for 30 min at room temperature. The reaction mixture is concentrated and used without further purification.

### Step 6 Preparation of 2-(3-(((2,4-diaminopteridin-6-yl)methyl)(methyl)amino)phenoxy)acetamide

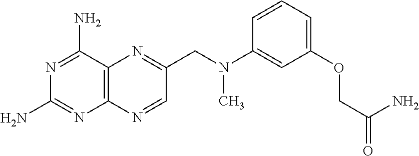

The title compound is prepared using a procedure similar to that of Example 3 except 2-(3-(methylamino)phenoxy)acetamide is used in place of 3-aminobenzoic acid. The residual material is dissolved in DMSO and purified by preparative HPLC (Method 2). Lyophilization of combined pure fractions affords desired product as a yellow solid (11 mg). NMR (400 MHz, DMSO-d₆) 3.14 (s, 3H), 4.39 (s, 2H), 4.77 (s, 2H), 6.26 (d, 1H), 6.37 (s, 6.41 (d, 1H), 7.07 (dd, 1H), 7.35 (br s, 1H), 7.45 (br s, 1H), 7.57 (br s, 1H), 8.58 (br s, 1H), 8.68 (s, 1H), 9.11 (s, 1H), 9.27 (s, 1H). LC-MS m/z 354.9 [M+H]⁺, retention time 3.46 min.

1. A compound of formula Q-I: 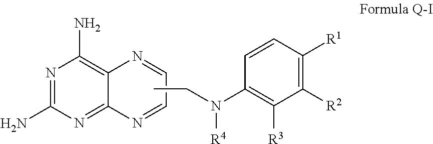
wherein
(i) R¹, R² and R³ are as follows:
(a) R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
(b) R² and R³ are independently COOR⁵; or
(c) R² and R³ are independently —OH or —OCH₂COOR⁵ and R¹ is —COOR⁵;
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
in free, salt or prodrug form.

2. The compound according to claim 1, wherein said compound is a compound of formula Q-II: 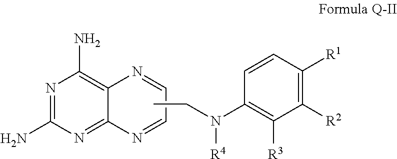
wherein
(i) R¹, R² and R³ are as follows:
(a) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵ or
(b) R² and R³ are independently —OH or —OCH₂COOR⁵ and R¹ is —COOR^{S};
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl),
(vi) Rₐ and R_{b} are independently H or C₁₋₄alkyl,
in free, salt or prodrug form.

3. The compound according to claim 1, wherein said compound is a compound of formula Q-III: 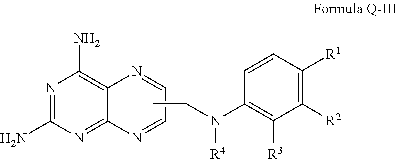
wherein
(i) R¹, R² and R³ are independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵, —C(O)N(Rₐ)(R_{b}), —OCH₂C(O)N(Rₐ)(R_{b}) or —C(H)(NH₂)COOR⁵;
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
in free, salt or prodrug form.

4. A compound of formula Q-IV: 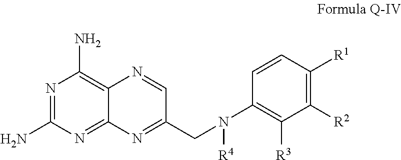
wherein
(i) R¹, R² and R³ independently H, —SO₂CF₃, —SO₃H, —NH₂, —C(O)N(R⁶)SO₃H, —CH₂C(O)COOR₅, —OH, —C₁₋₄alkyl-COOR⁵ (e.g., —CH₂COOR⁵), —P(O)(OR⁵)₂, —C₁₋₄alkyl-P(O)(OR⁵)₂ (e.g., —CH₂P(O)(OR⁵)₂), —OCH₂COOR⁵; —C(O)N(R⁶)S(O)₂R⁷, —C(H)═C(OH)C(O)OR⁵′, —C(H)(NH₂)COOR⁵, —COOR^{S}, C(O)N(Rₐ)(R_{b}) or —OCH₂C(O)N(Rₐ)(R_{b});
(ii) R⁴ is a H or C₁₋₄alkyl (e.g., methyl);
(iii) R⁵ is a H or C₁₋₄alkyl (e.g., ethyl);
(iv) R⁶ is H or C₁₋₄alkyl;
(v) R⁷ is C₁₋₄alkyl (e.g., methyl);
(vi) Rₐ and R_{b} are independently H or C₁₋₄alkyl,
in free, salt or prodrug form.

5. The compound according to claim 1, wherein the compound is selected from a group consisting of: 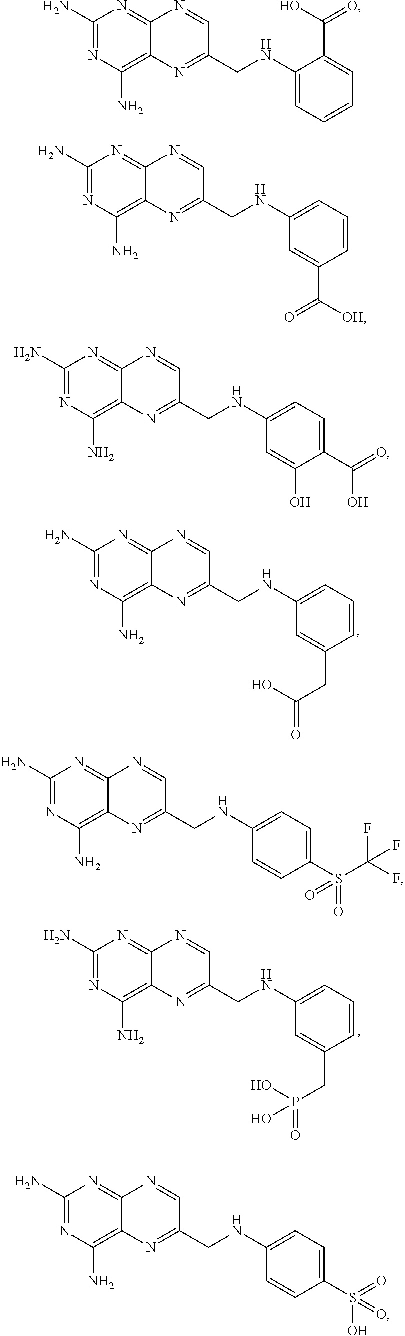 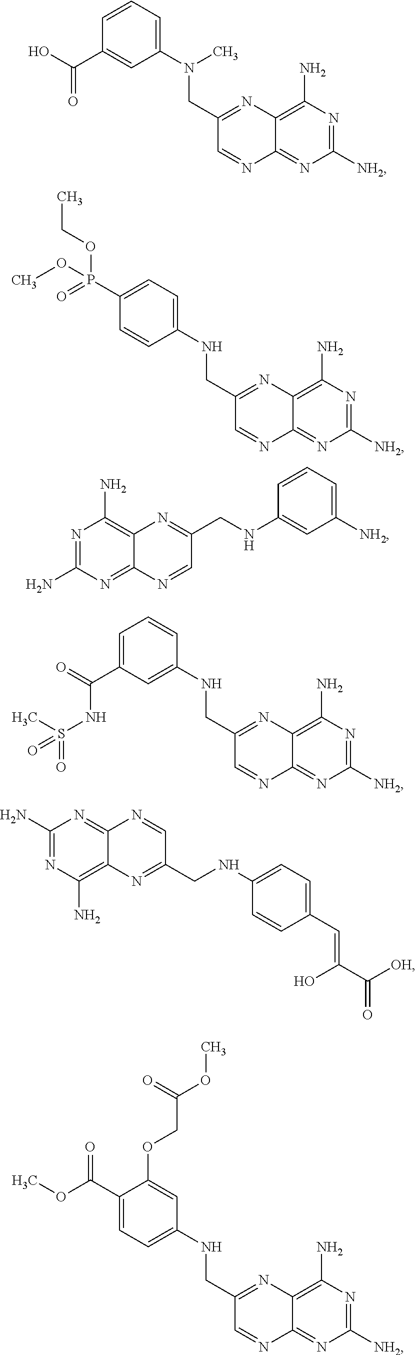 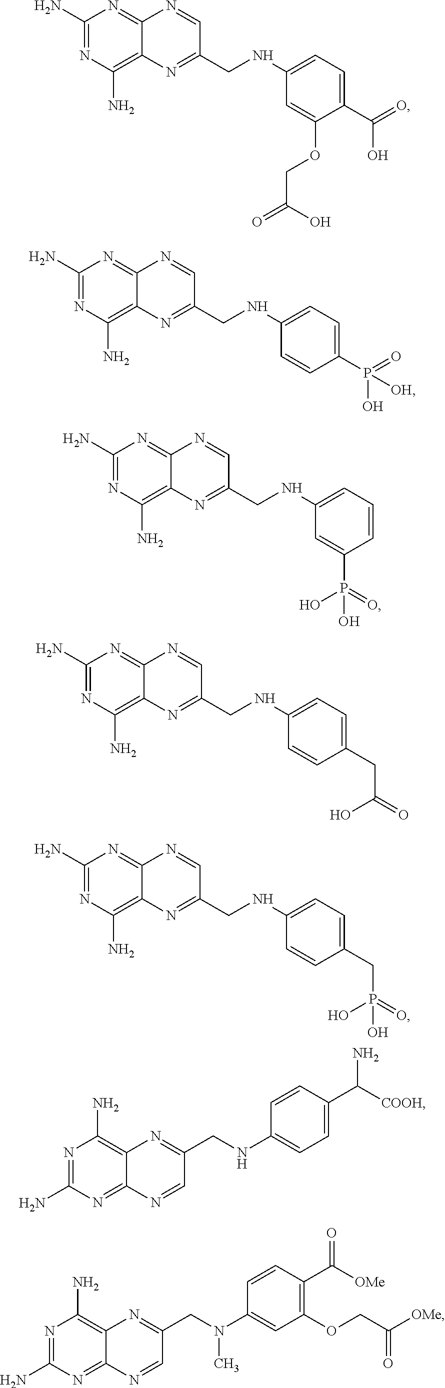 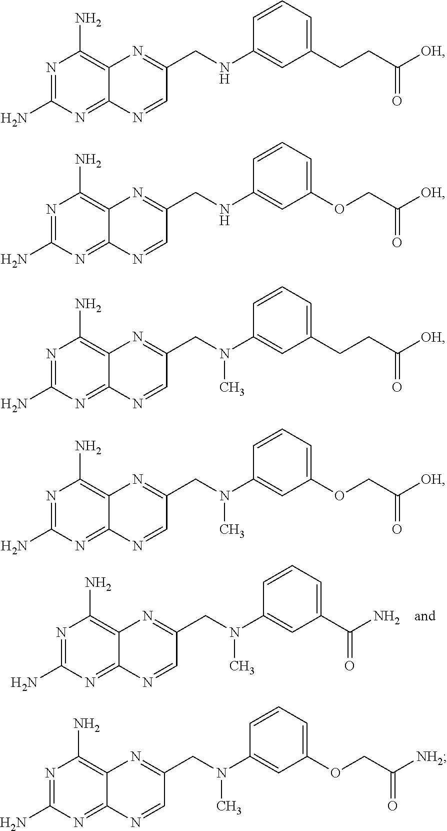
in free, salt or prodrug form.

6. A compound of formula V: 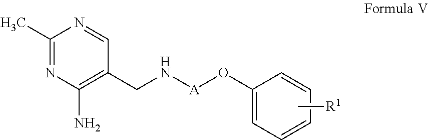
wherein:
A is a heteroaryl, e.g., 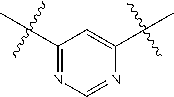
R¹ is C₁₋₄alkyl-COOR² (e.g, —CH₂COOR²)
R² is H or C₁₋₄alkyl (e.g., methyl);
in free, salt or prodrug form.

7. A compound of formula VI: 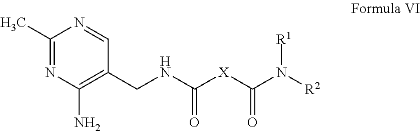
wherein:
X is a C₁₋₄alkyl, e.g., ethyl;
R¹ and R² are independently H or C₁₋₈alkyl-P(O)(OR³)(OR⁴), e.g, —CH₂CH₂P(O)(OR³)(OR⁴);
R³ and R⁴ are independently H, C₁₋₄alkyl (e.g., ethyl) or C₁₋₄alkyl-OC(O)R⁵ (e.g., CH₂CH₂OC(O)R⁵;
R⁵ is C₁₋₄alkyl (e.g., t-butyl),
in free, salt or prodrug form.

8. A method for the treatment or prophylaxis of an infection comprising administering to a subject in need thereof an effective amount of a compound according to claim 1, in free, pharmaceutically acceptable salt or prodrug form.

9. The method according to any of claim 8, wherein the infection is a Gram-positive or Gram-negative bacterial infection.

10. The method according to claim 8, wherein the bacterial infection is selected from a group consisting of Moraxella catarrhalis, Klebsiella pneumoniae, Staphylococcus epidermidis, Streptococcus viridans, Enterococcus faecium, Staphylococcus aureus, Bacillus anthracia, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi.

11. The method according to claim 8, wherein the bacterial infection is a Staphylococcus aureus infection.

12. The method according to claim 8, wherein the compound is selected from: 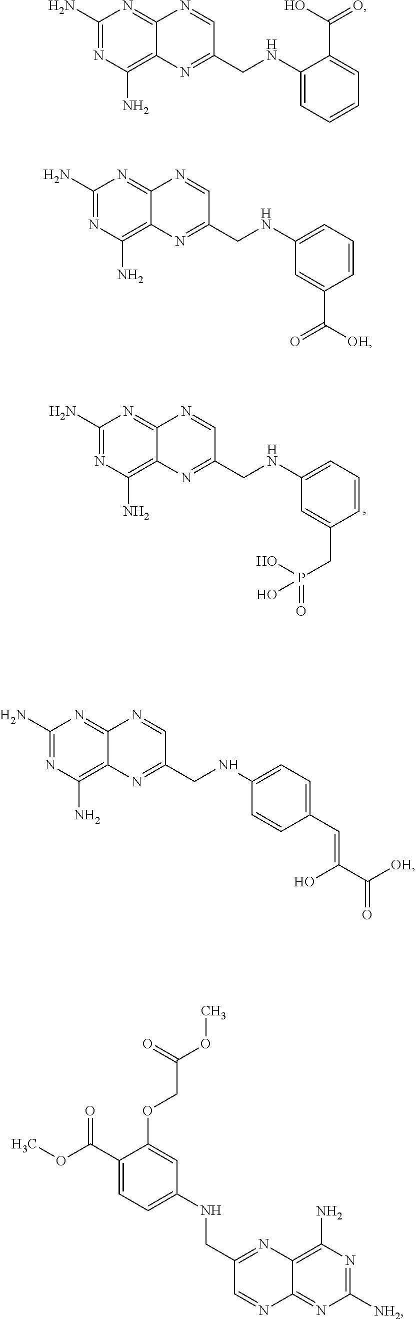 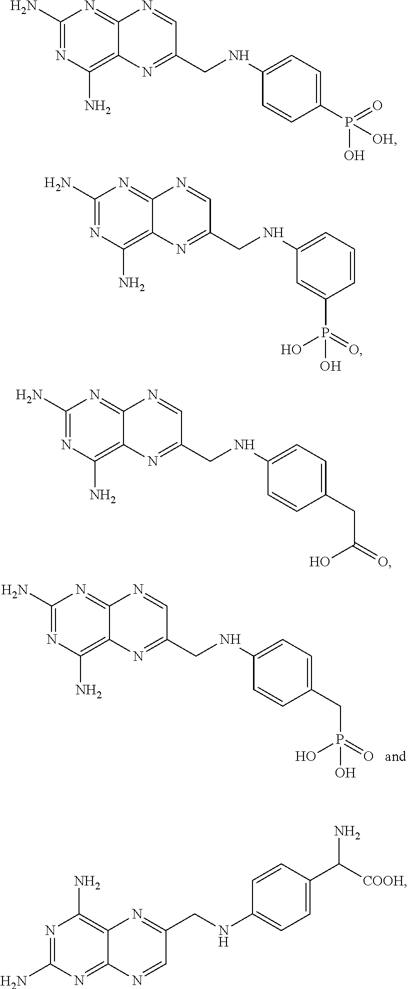
in free, pharmaceutically acceptable salt or prodrug form.

15. The method according to claim 14, wherein said infection is by an infectious agent which is resistant to a drug that is not a riboswitch ligand.

16. The method according to claim 14, wherein the infection is an infection which is resistant to one or more drugs selected from a group consisting of a penicillin, vancomycin, cephalosporin and methicillin.

17. The method according to claim 14, wherein the infection is a methicillin-resistant Staphylococcus aureus infection.

18. A pharmaceutical composition comprising a compound as described in claim 1, in free, pharmaceutically acceptable salt or prodrug form, in admixture with a pharmaceutically acceptable diluent or carrier.

19. (canceled)

20. (canceled)

21. (canceled)

22. (canceled)

23. A method for the treatment or prophylaxis of a fungal infection comprising administering to a subject in need thereof an effective amount of a compound as described in claim 1, in free, pharmaceutically acceptable salt or prodrug form, or a pharmaceutical composition as described in claim 18.

24. A method for the treatment or prophylaxis of a condition, disease or infection selected from anthrax, staphylococcal scalded skin syndrome (staph infections), lyme disease, pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound as described in claim 1, in free, pharmaceutically acceptable salt or prodrug form, or a pharmaceutical composition as described in claim 18.
